SEQUENCE LISTING (51 sequences)

SEQ ID NO: 1 - protein, 4 aa, Artificial Sequence
  linker or affinity tag
SVEA

SEQ ID NO: 2 - protein, 4 aa, Artificial Sequence
  linker or affinity tag
DMDF

SEQ ID NO: 3 - protein, 453 aa, Artificial Sequence
  recombinant protein
MGAAARTLRLALGLLLLATLLRPADASSAPHHHHHHDAGGRPRSSPDDLKALTRNVNRLNESFRDLQLRLLQAPLQADLTEQVWKVQDALQNQSDSLLALAGAVQRLEGALWGLQAQAVQTEQAVALLRDRTGQQSDTAQLELYQLQVESNSSQLLLRRHAGLLDGLARRVGILGEELADVGGVLRGLNHSLSYDVALHRTRLQDLRVLVSNASEDTRRLRLAHVGMELQLKQELAMLNAVTEDLRLKDWEHSIALRNISLAKGPPGPKGDQGDEGKEGRPGIPGLPGLRGLPGERGTPGLPGPKGDDGKLGATGPMGMRGFKGDRGPKGEKGEKGDRAGDASGVEAPMMIRLVNGSGPHEGRVEVYHDRRWGTVCDDGWDKKDGDVVCRMLGFRGVEEVYRTARFGQGTGRIWMDDVACKGTEETIFRCSFSKWGVTNCGHAEDASVTCNRH

SEQ ID NO: 4 - protein, 195 aa, Artificial Sequence
  recombinant protein
MGAAARTLRLALGLLLLATLLRPADASSAPHHHHHHDAGGPGLPGPKGDDGKLGATGPMGMRGFKGDRGPKGEKGEKGDRAGDASGVEAPMMIRLVNGSGPHEGRVEVYHDRRWGTVCDDGWDKKDGDVVCRMLGFRGVEEVYRTARFGQGTGRIWMDDVACKGTEETIFRCSFSKWGVTNCGHAEDASVTCNRH

SEQ ID NO: 5 - protein, 449 aa, Artificial Sequence
  recombinant protein
MGAAARTLRLALGLLLLATLLRPADASSAPHHHHHHDAGGRPRSSPDDLKALTRNVNRLNESLRDMQLRLLQAPLQADLTEQVWKVQDALQNQTDSLLALAGLVQRLEGTLWGLHAQAAQTEQATALLRDRTGQQSDSAQLELYQLQVESNRSQLLLQRHAGLLDGLARRVGVLGEELADVGGALRGLNHSLSYDVALHSTWLQDLQVLVSNASADTRRMRLVHMDMEMQLKQELATLNVVTEDLRLKDWEHSIALRNITLAKGPPGPKGDQGNEGKEGKPGSPGLPGSRGLPGERGDPGLPGPKGDDGKLGATGPMGMRGFKGDRGPKGEKGERGERAGDMDFTMIRLVNGSGPHQGRVEVFHDRRWGTVCDDGWDKKDGDVVCRMLGFHGVEEVYRTARFGQGTGRIWMDDVNCKGTESSIFHCQFSKWGVTNCGHAEDAGVTCTVP

SEQ ID NO: 6 - protein, 191 aa, Artificial Sequence
  recombinant protein
MGAAARTLRLALGLLLLATLLRPADASSAPHHHHHHDAGGPGLPGPKGDDGKLGATGPMGMRGFKGDRGPKGEKGERGERAGDMDFTMIRLVNGSGPHQGRVEVFHDRRWGTVCDDGWDKKDGDVVCRMLGFHGVEEVYRTARFGQGTGRIWMDDVNCKGTESSIFHCQFSKWGVTNCGHAEDAGVTCTVP

SEQ ID NO: 7 - protein, 355 aa, Artificial Sequence
  recombinant protein
MRAWIFFLLCLAGRALAAPLAHHHHHHHHKLQEQLQMLEMCCGNGSLAIEDKPFFSLQWAPKTHLVPRAQGLQALQAQLSWVHTSQEQLRQQFNNLTQNPELFQIKGERGSPGPKGAPGAPGIPGLPGPAAEKGEKGAAGRDGTPGVQGPQGPPGSKGEAGLQGLTGAPGKQGATGAPGPRGEKGSKGDIGLTGPKGEHGTKGDKGDLGLPGNKGDMGMKGDTGPMGSPGAQGGKGDAGKPGLPGLAGSPGVKGDQFQRVRIMGGTNRGRAEVYYNNEWGTICDDDWDNNDATVFCRMLGYSRGRALSSYGGGSGNIWLDNVNCRGTENSLWDCSKNSWGNHNCVHNEDAGVECS

SEQ ID NO: 8 - protein, 370 aa, Artificial Sequence
  recombinant protein
MGAAARTLRLALGLLLLATLLRPADASSAPHHHHHHDAGGQARLRVLEMYFLNDTLAAEDSPSFSLLQSAHPGEHLAQGASRLQVLQAQLTWVRVSHEHLLQRVDNFTQNPGMFRIKGEQGAPGLQGHKGAMGMPGAPGPPGPPAEKGAKGAMGRDGATGPSGPQGPPGVKGEAGLQGPQGAPGKQGATGTPGPQGEKGSKGDGGLIGPKGETGTKGEKGDLGLPGSKGDRGMKGDAGVMGPPGAQGSKGDFGRPGPPGLAGFPGAKGDQSVSVRIVGSSNRGRAEVYYSGTWGTICDDEWQNSDAIVFCRMLGYSKGRALYKVGAGTGQIWLDNVQCRGTESTLWSCTKNSWGHHDCSHEEDAGVECSV

SEQ ID NO: 9 - protein, 448 aa, Artificial Sequence
  recombinant protein
MGAAARTLRLALGLLLLATLLRPADASSAPHHHHHHDAGGRPRSSPDDLKALTRNVNRLNESLRDMQLRLLQAPLQADLTEQVWKVQDALQNQTDSLLALAGLVQRLEGTLWGLHAQAAQTEQAMALLRDRTGQQSDSAQLELYQLQVESNRSQLLLQRHAGLLDGLARRVGVLGEELADVGGALRGLNHSLSYDVALHSTWLQDLQVLVSNASADTRRMRLVHMDMEMQLKQELATLNVVTEDLRLKDWEHSIALRNITLAKGPPGPKGDQGNEGKEGKPGSPGLPGSRGLPGERGDPGLPGPKGDDGKLGATGPMGMRGFKGDRGPKGEKGERGERGGSTPLKTVRLVGGSGAHEGRVEIFHQGQWGTICDDRWDIRAGQVVCRSLGYQEVLAVHKRAHFGQGTGPIWLNEVMCFGRESSIENCKINQWGVLSCSHSEDAGVTCTS

SEQ ID NO: 10 - protein, 448 aa, Artificial Sequence
  recombinant protein
MGAAARTLRLALGLLLLATLLRPADASSAPHHHHHHDAGGRPRSSPDDLKALTRNVNRLNESFRDLQLRLLQAPLQADLTEQVWKVQDALQNQSDSLLALAGAVQRLEGALWGLQAQAVQTEQAVALLRDRTGQQSDTAQLELYQLQVESNSSQLLLRRHAGLLDGLARRVGILGEELADVGGVLRGLNHSLSYDVALHRTRLQDLRVLVSNASEDTRRLRLAHVGMELQLKQELAMLNAVTEDLRLKDWEHSIALRNISLAKGPPGPKGDQGDEGKEGRPGIPGLPGLRGLPGERGTPGLPGPKGDDGKLGATGPMGMRGFKGDRGPKGEKGEKGDRNTLTPFTKVRLVGGSGPHEGRVEILHSGQWGTICDDRWEVRVGQVVCRSLGYPGVQAVHKAAHFGQGTGPIWLNEVFCFGRESSIEECKIRQWGTRACSHSEDAGVTCTL

SEQ ID NO: 11 - protein, 5 aa, Artificial Sequence
  linker or affinity tag
GAAGG

SEQ ID NO: 12 - protein, 5 aa, Artificial Sequence
  linker or affinity tag
AAAGG

SEQ ID NO: 13 - protein, 4 aa, Artificial Sequence
  linker or affinity tag
DAGG

SEQ ID NO: 14 - protein, 3 aa, Artificial Sequence
  linker or affinity tag
HHK

SEQ ID NO: 15 - protein, 30 aa, Homo sapiens
MGAAARTLRLALGLLLLATLLRPADASSAP

SEQ ID NO: 16 - protein, 21 aa, Homo sapiens
MRAWIFFLLCLAGRALAAPLA

SEQ ID NO: 17 - protein, 413 aa, Artificial Sequence
  recombinant protein
RPRSSPDDLKALTRNVNRLNESFRDLQLRLLQAPLQADLTEQVWKVQDALQNQSDSLLALAGAVQRLEGALWGLQAQAVQTEQAVALLRDRTGQQSDTAQLELYQLQVESNSSQLLLRRHAGLLDGLARRVGILGEELADVGGVLRGLNHSLSYDVALHRTRLQDLRVLVSNASEDTRRLRLAHVGMELQLKQELAMLNAVTEDLRLKDWEHSIALRNISLAKGPPGPKGDQGDEGKEGRPGIPGLPGLRGLPGERGTPGLPGPKGDDGKLGATGPMGMRGFKGDRGPKGEKGEKGDRAGDASGVEAPMMIRLVNGSGPHEGRVEVYHDRRWGTVCDDGWDKKDGDVVCRMLGFRGVEEVYRTARFGQGTGRIWMDDVACKGTEETIFRCSFSKWGVTNCGHAEDASVTCNRH

SEQ ID NO: 18 - protein, 155 aa, Artificial Sequence
  recombinant protein
PGLPGPKGDDGKLGATGPMGMRGFKGDRGPKGEKGEKGDRAGDASGVEAPMMIRLVNGSGPHEGRVEVYHDRRWGTVCDDGWDKKDGDVVCRMLGFRGVEEVYRTARFGQGTGRIWMDDVACKGTEETIFRCSFSKWGVTNCGHAEDASVTCNRH

SEQ ID NO: 19 - protein, 409 aa, Artificial Sequence
  recombinant protein
RPRSSPDDLKALTRNVNRLNESLRDMQLRLLQAPLQADLTEQVWKVQDALQNQTDSLLALAGLVQRLEGTLWGLHAQAAQTEQATALLRDRTGQQSDSAQLELYQLQVESNRSQLLLQRHAGLLDGLARRVGVLGEELADVGGALRGLNHSLSYDVALHSTWLQDLQVLVSNASADTRRMRLVHMDMEMQLKQELATLNVVTEDLRLKDWEHSIALRNITLAKGPPGPKGDQGNEGKEGKPGSPGLPGSRGLPGERGDPGLPGPKGDDGKLGATGPMGMRGFKGDRGPKGEKGERGERAGDMDFTMIRLVNGSGPHQGRVEVFHDRRWGTVCDDGWDKKDGDVVCRMLGFHGVEEVYRTARFGQGTGRIWMDDVNCKGTESSIFHCQFSKWGVTNCGHAEDAGVTCTVP

SEQ ID NO: 20 - protein, 151 aa, Artificial Sequence
  recombinant protein
PGLPGPKGDDGKLGATGPMGMRGFKGDRGPKGEKGERGERAGDMDFTMIRLVNGSGPHQGRVEVFHDRRWGTVCDDGWDKKDGDVVCRMLGFHGVEEVYRTARFGQGTGRIWMDDVNCKGTESSIFHCQFSKWGVTNCGHAEDAGVTCTVP

SEQ ID NO: 21 - protein, 325 aa, Artificial Sequence
  recombinant protein
LQEQLQMLEMCCGNGSLAIEDKPFFSLQWAPKTHLVPRAQGLQALQAQLSWVHTSQEQLRQQFNNLTQNPELFQIKGERGSPGPKGAPGAPGIPGLPGPAAEKGEKGAAGRDGTPGVQGPQGPPGSKGEAGLQGLTGAPGKQGATGAPGPRGEKGSKGDIGLTGPKGEHGTKGDKGDLGLPGNKGDMGMKGDTGPMGSPGAQGGKGDAGKPGLPGLAGSPGVKGDQFQRVRIMGGTNRGRAEVYYNNEWGTICDDDWDNNDATVFCRMLGYSRGRALSSYGGGSGNIWLDNVNCRGTENSLWDCSKNSWGNHNCVHNEDAGVECS

SEQ ID NO: 22 - protein, 9 aa, Artificial Sequence
  linker or affinity tag
YPYDVPDYA

SEQ ID NO: 23 - protein, 14 aa, Artificial Sequence
  linker or affinity tag
GKPIPNPLLGLDST

SEQ ID NO: 24 - protein, 10 aa, Artificial Sequence
  linker or affinity tag
EQKLISEEDL

SEQ ID NO: 25 - protein, 9 aa, Artificial Sequence
  linker or affinity tag
DYKDDDDKG

SEQ ID NO: 26 - protein, 11 aa, Artificial Sequence
  linker or affinity tag
QPELAPEDPED

SEQ ID NO: 27 - protein, 11 aa, Artificial Sequence
  linker or affinity tag
YTDIEMNRLGK

SEQ ID NO: 28 - protein, 6 aa, Artificial Sequence
  linker or affinity tag
HHHHHH

SEQ ID NO: 29 - protein, 11 aa, Artificial Sequence
  linker or affinity tag
ASMTGGQQMGR

SEQ ID NO: 30 - protein, 70 aa, Artificial Sequence
  linker or affinity tag
GAGTPVTAPLAGTIWKVLSEGQTVAAGEVLLILEAMKETEIRAAQAGTVRGIAVKAGDAVAVGDTLMTLA

SEQ ID NO: 31 - protein, 8 aa, Artificial Sequence
  linker or affinity tag
WSHPQFEK

SEQ ID NO: 32 - protein, 330 aa, Artificial Sequence
  recombinant protein
QARLRVLEMYFLNDTLAAEDSPSFSLLQSAHPGEHLAQGASRLQVLQAQLTWVRVSHEHLLQRVDNFTQNPGMFRIKGEQGAPGLQGHKGAMGMPGAPGPPGPPAEKGAKGAMGRDGATGPSGPQGPPGVKGEAGLQGPQGAPGKQGATGTPGPQGEKGSKGDGGLIGPKGETGTKGEKGDLGLPGSKGDRGMKGDAGVMGPPGAQGSKGDFGRPGPPGLAGFPGAKGDQSVSVRIVGSSNRGRAEVYYSGTWGTICDDEWQNSDAIVFCRMLGYSKGRALYKVGAGTGQIWLDNVQCRGTESTLWSCTKNSWGHHDCSHEEDAGVECSV

SEQ ID NO: 33 - protein, 408 aa, Artificial Sequence
  recombinant protein
RPRSSPDDLKALTRNVNRLNESLRDMQLRLLQAPLQADLTEQVWKVQDALQNQTDSLLALAGLVQRLEGTLWGLHAQAAQTEQAMALLRDRTGQQSDSAQLELYQLQVESNRSQLLLQRHAGLLDGLARRVGVLGEELADVGGALRGLNHSLSYDVALHSTWLQDLQVLVSNASADTRRMRLVHMDMEMQLKQELATLNVVTEDLRLKDWEHSIALRNITLAKGPPGPKGDQGNEGKEGKPGSPGLPGSRGLPGERGDPGLPGPKGDDGKLGATGPMGMRGFKGDRGPKGEKGERGERGGSTPLKTVRLVGGSGAHEGRVEIFHQGQWGTICDDRWDIRAGQVVCRSLGYQEVLAVHKRAHFGQGTGPIWLNEVMCFGRESSIENCKINQWGVLSCSHSEDAGVTCTS

SEQ ID NO: 34 - protein, 408 aa, Artificial Sequence
  recombinant protein
RPRSSPDDLKALTRNVNRLNESFRDLQLRLLQAPLQADLTEQVWKVQDALQNQSDSLLALAGAVQRLEGALWGLQAQAVQTEQAVALLRDRTGQQSDTAQLELYQLQVESNSSQLLLRRHAGLLDGLARRVGILGEELADVGGVLRGLNHSLSYDVALHRTRLQDLRVLVSNASEDTRRLRLAHVGMELQLKQELAMLNAVTEDLRLKDWEHSIALRNISLAKGPPGPKGDQGDEGKEGRPGIPGLPGLRGLPGERGTPGLPGPKGDDGKLGATGPMGMRGFKGDRGPKGEKGEKGDRNTLTPFTKVRLVGGSGPHEGRVEILHSGQWGTICDDRWEVRVGQVVCRSLGYPGVQAVHKAAHFGQGTGPIWLNEVFCFGRESSIEECKIRQWGTRACSHSEDAGVTCTL

SEQ ID NO: 35 - protein, 470 aa, Artificial Sequence [sequence omitted]

SEQ ID NO: 36 - protein, 480 aa, Artificial Sequence
  recombinant protein
MGAAARTLRLALGLLLLATLLRPADASSAAWRHPQFGGGSGGGSGGGSAWRHPQFGGSGHHHHHHHHRPRSSPDDLKALTRNVNRLNESFRDLQLRLLQAPLQADLTEQVWKVQDALQNQSDSLLALAGAVQRLEGALWGLQAQAVQTEQAVALLRDRTGQQSDTAQLELYQLQVESNSSQLLLRRHAGLLDGLARRVGILGEELADVGGVLRGLNHSLSYDVALHRTRLQDLRVLVSNASEDTRRLRLAHVGMELQLKQELAMLNAVTEDLRLKDWEHSIALRNISLAKGPPGPKGDQGDEGKEGRPGIPGLPGLRGLPGERGTPGLPGPKGDDGKLGATGPMGMRGFKGDRGPKGEKGEKGDRAGDASGVEAPMMIRLVNGSGPHEGRVEVYHDRRWGTVCDDGWDKKDGDVVCRMLGFRGVEEVYRTARFGQGTGRIWMDDVACKGTEETIFRCSFSKWGVTNCGHAEDASVTCNRH

SEQ ID NO: 37 - protein, 387 aa, Artificial Sequence
  recombinant protein
MGAAARTLRLALGLLLLATLLRPADASSAPHHHHHHHHSGGLNDIFEAQKIEWHESGQARLRVLEMYFLNDTLAAEDSPSFSLLQSAHPGEHLAQGASRLQVLQAQLTWVRVSHEHLLQRVDNFTQNPGMFRIKGEQGAPGLQGHKGAMGMPGAPGPPGPPAEKGAKGAMGRDGATGPSGPQGPPGVKGEAGLQGPQGAPGKQGATGTPGPQGEKGSKGDGGLIGPKGETGTKGEKGDLGLPGSKGDRGMKGDAGVMGPPGAQGSKGDFGRPGPPGLAGFPGAKGDQSVSVRIVGSSNRGRAEVYYSGTWGTICDDEWQNSDAIVFCRMLGYSKGRALYKVGAGTGQIWLDNVQCRGTESTLWSCTKNSWGHHDCSHEEDAGVECSV

SEQ ID NO: 38 - protein, 397 aa, Artificial Sequence
  recombinant protein
MGAAARTLRLALGLLLLATLLRPADASSAAWRHPQFGGGSGGGSGGGSAWRHPQFGGSGHHHHHHHHQARLRVLEMYFLNDTLAAEDSPSFSLLQSAHPGEHLAQGASRLQVLQAQLTWVRVSHEHLLQRVDNFTQNPGMFRIKGEQGAPGLQGHKGAMGMPGAPGPPGPPAEKGAKGAMGRDGATGPSGPQGPPGVKGEAGLQGPQGAPGKQGATGTPGPQGEKGSKGDGGLIGPKGETGTKGEKGDLGLPGSKGDRGMKGDAGVMGPPGAQGSKGDFGRPGPPGLAGFPGAKGDQSVSVRIVGSSNRGRAEVYYSGTWGTICDDEWQNSDAIVFCRMLGYSKGRALYKVGAGTGQIWLDNVQCRGTESTLWSCTKNSWGHHDCSHEEDAGVECSV

SEQ ID NO: 39 - protein, 387 aa, Artificial Sequence
  recombinant protein
MGAAARTLRLALGLLLLATLLRPADASSAPHHHHHHHHSGGLNDIFEAQKIEWHESGQARLRVLEMYFLNDTLAAEDSPSFSLLQSAHPGEHLAQGASRLQVLQAQLTWVRVSHEHLLQRVDNFTQNPGMFRIKGEQGAPGLQGHKGAMGMPGAPGPPGPPAEKGAKGAMGRDGATGPSGPQGPPGVKGEAGLQGPQGAPGLPGSPGSPGATGLKGSKGDTGLQGQQGRKGESGVPGPAGVKGEQGSPGLAGPKGAPGQAGQKGDQGVKGSSGEQGVKGEKGERGENSVSVRIVGSSNRGRAEVYYSGTWGTICDDEWQNSDAIVFCRMLGYSKGRALYKVGAGTGQIWLDNVQCRGTESTLWSCTKNSWGHHDCSHEEDAGVECSV

SEQ ID NO: 40 - protein, 397 aa, Artificial Sequence
  recombinant protein
MGAAARTLRLALGLLLLATLLRPADASSAAWRHPQFGGGSGGGSGGGSAWRHPQFGGSGHHHHHHHHQARLRVLEMYFLNDTLAAEDSPSFSLLQSAHPGEHLAQGASRLQVLQAQLTWVRVSHEHLLQRVDNFTQNPGMFRIKGEQGAPGLQGHKGAMGMPGAPGPPGPPAEKGAKGAMGRDGATGPSGPQGPPGVKGEAGLQGPQGAPGLPGSPGSPGATGLKGSKGDTGLQGQQGRKGESGVPGPAGVKGEQGSPGLAGPKGAPGQAGQKGDQGVKGSSGEQGVKGEKGERGENSVSVRIVGSSNRGRAEVYYSGTWGTICDDEWQNSDAIVFCRMLGYSKGRALYKVGAGTGQIWLDNVQCRGTESTLWSCTKNSWGHHDCSHEEDAGVECSV

SEQ ID NO: 41 - protein, 463 aa, Artificial Sequence
  recombinant protein
MGAAARTLRLALGLLLLATLLRPADASSAPHHHHHHHHSGGLNDIFEAQKIEWHERPRSSPDDLKALTRNVNRLNESFRDLQLRLLQAPLQADLTEQVWKVQDALQNQSDSLLALAGAVQRLEGALWGLQAQAVQTEQAVALLRDRTGQQSDTAQLELYQLQVESNSSQLLLRRHAGLLDGLARRVGILGEELADVGGVLRGLNHSLSYDVALHRTRLQDLRVLVSNASEDTRRLRLAHVGMELQLKQELAMLNAVTEDLRLKDWEHSIALRNISLAKGPPGPKGDQGDEGKEGRPGIPGLPGLRGLPGERGTPGLPGPKGDDGKLGATGPMGMRGFKGDRGPKGEKGEKGDRNTLTPFTKVRLVGGSGPHEGRVEILHSGQWGTICDDRWEVRVGQVVCRSLGYPGVQAVHKAAHFGQGTGPIWLNEVFCFGRESSIEECKIRQWGTRACSHSEDAGVTCTL

SEQ ID NO: 42 - protein, 475 aa, Artificial Sequence
  recombinant protein
MGAAARTLRLALGLLLLATLLRPADASSAAWRHPQFGGGSGGGSGGGSAWRHPQFGGSGHHHHHHHHRPRSSPDDLKALTRNVNRLNESFRDLQLRLLQAPLQADLTEQVWKVQDALQNQSDSLLALAGAVQRLEGALWGLQAQAVQTEQAVALLRDRTGQQSDTAQLELYQLQVESNSSQLLLRRHAGLLDGLARRVGILGEELADVGGVLRGLNHSLSYDVALHRTRLQDLRVLVSNASEDTRRLRLAHVGMELQLKQELAMLNAVTEDLRLKDWEHSIALRNISLAKGPPGPKGDQGDEGKEGRPGIPGLPGLRGLPGERGTPGLPGPKGDDGKLGATGPMGMRGFKGDRGPKGEKGEKGDRNTLTPFTKVRLVGGSGPHEGRVEILHSGQWGTICDDRWEVRVGQVVCRSLGYPGVQAVHKAAHFGQGTGPIWLNEVFCFGRESSIEECKIRQWGTRACSHSEDAGVTCTL

SEQ ID NO: 43 - protein, 432 aa, Artificial Sequence
  recombinant protein
MGAAARTLRLALGLLLLATLLRPADASSAPHHHHHHHHSGGLNDIFEAQKIEWHESGKWETKNCSVSSTNANDITQSLTGKGNDSEEEMRFQEVFMEHMSNMEKRIQHILDMEANLMDTEHFQNFSMTTDQRFNDILLQLSTLFSSVQGHGNAIDEISKSLISLNTTLLDLQLNIENLNGKIQENTFKQQEEISKLEERVYNVSAEIMAMKEEQVHLEQEIKGEVKVLNNITNDLRLKDWEHSQTLRNITLIQGPPGPPGEKGDRGPTGESGPRGFPGPIGPPGLKGDRGAIGFPGSRGLPGYAGRPGNSGPKGQKGEKGSGNTLTPFTKVRLVGGSGPHEGRVEILHSGQWGTICDDRWEVRVGQVVCRSLGYPGVQAVHKAAHFGQGTGPIWLNEVFCFGRESSIEECKIRQWGTRACSHSEDAGVTCTL

SEQ ID NO: 44 - protein, 442 aa, Artificial Sequence
  recombinant protein
MGAAARTLRLALGLLLLATLLRPADASSAAWRHPQFGGGSGGGSGGGSAWRHPQFGGSGHHHHHHHHKWETKNCSVSSTNANDITQSLTGKGNDSEEEMRFQEVFMEHMSNMEKRIQHILDMEANLMDTEHFQNFSMTTDQRFNDILLQLSTLFSSVQGHGNAIDEISKSLISLNTTLLDLQLNIENLNGKIQENTFKQQEEISKLEERVYNVSAEIMAMKEEQVHLEQEIKGEVKVLNNITNDLRLKDWEHSQTLRNITLIQGPPGPPGEKGDRGPTGESGPRGFPGPIGPPGLKGDRGAIGFPGSRGLPGYAGRPGNSGPKGQKGEKGSGNTLTPFTKVRLVGGSGPHEGRVEILHSGQWGTICDDRWEVRVGQVVCRSLGYPGVQAVHKAAHFGQGTGPIWLNEVFCFGRESSIEECKIRQWGTRACSHSEDAGVTCTL

SEQ ID NO: 45 - protein, 15 aa, Artificial Sequence
  linker or affinity tag
GLNDIFEAQKIEWHE

SEQ ID NO: 46 - protein, 606 aa, Homo sapiens
MKVRSAGGDGDALCVTEEDLAGDDEDMPTFPCTQKGRPGPRCSRCQKNLSLHTSVRILYLFLALLLVAVAVLASLVFRKVDSLSEDISLTQSIYDKKLVLMQKNLQGLDPKALNNCSFCHEAGQLGPEIRKLQEELEGIQKLLLAQEVQLDQTLQAQEVLSTTSRQISQEMGSCSFSIHQVNQSLGLFLAQVRGWQATTAGLDLSLKDLTQECYDVKAAVHQINFTVGQTSEWIHGIQRKTDEETLTLQKIVTDWQNYTRLFSGLRTTSTKTGEAVKNIQATLGASSQRISQNSESMHDLVLQVMGLQLQLDNISSFLDDHEENMHDLQYHTHYAQNRTVERFESLEGRMASHEIEIGTIFTNINATDNHVHSMLKYLDDVRLSCTLGFHTHAEELYYLNKSVSIMLGTTDLLRERFSLLSARLDLNVRNLSMIVEEMKAVDTQHGEILRNVTILRGAPGPPGPRGFKGDMGVKGPVGGRGPKGDPGSLGPLGPQGPQGQPGEAGPVGERGPVGPRGFPGLKGSKGSFGTGGPRGQPGPKGDIGPPGPEGPPGSPGPSGPQGKPGIAGKTGSPGQRGAMGPKGEPGIQGPPGLPGPPGPPGSQSFY

SEQ ID NO: 47 - protein, 472 aa, Homo sapiens
MGCDRNCGLIAGAVIGAVLAVFGGILMPVGDLLIQKTIKKQVVLEEGTIAFKNWVKTGTEVYRQFWIFDVQNPQEVMMNSSNIQVKQRGPYTYRVRFLAKENVTQDAEDNTVSFLQPNGAIFEPSLSVGTEADNFTVLNLAVAAASHIYQNQFVQMILNSLINKSKSSMFQVRTLRELLWGYRDPFLSLVPYPVTTTVGLFYPYNNTADGVYKVFNGKDNISKVAIIDTYKGKRNLSYWESHCDMINGTDAASFPPFVEKSQVLQFFSSDICRSIYAVFESDVNLKGIPVYRFVLPSKAFASPVENPDNYCFCTEKIISKNCTSYGVLDISKCKEGRPVYISLPHFLYASPDVSEPIDGLNPNEEEHRTYLDIEPITGFTLQFAKRLQVNLLVKPSEKIQVLKNLKRNYIVPILWLNETGTIGDEKANMFRSQVTGKINLLGLIEMILLSVGVVMFVAFMISYCACRSKTIK

SEQ ID NO: 48 - protein, 1156 aa, Homo sapiens
MSKLRMVLLEDSGSADFRRHFVNLSPFTITVVLLLSACFVTSSLGGTDKELRLVDGENKCSGRVEVKVQEEWGTVCNNGWSMEAVSVICNQLGCPTAIKAPGWANSSAGSGRIWMDHVSCRGNESALWDCKHDGWGKHSNCTHQQDAGVTCSDGSNLEMRLTRGGNMCSGRIEIKFQGRWGTVCDDNFNIDHASVICRQLECGSAVSFSGSSNFGEGSGPIWFDDLICNGNESALWNCKHQGWGKHNCDHAEDAGVICSKGADLSLRLVDGVTECSGRLEVRFQGEWGTICDDGWDSYDAAVACKQLGCPTAVTAIGRVNASKGFGHIWLDSVSCQGHEPAIWQCKHHEWGKHYCNHNEDAGVTCSDGSDLELRLRGGGSRCAGTVEVEIQRLLGKVCDRGWGLKEADVVCRQLGCGSALKTSYQVYSKIQATNTWLFLSSCNGNETSLWDCKNWQWGGLTCDHYEEAKITCSAHREPRLVGGDIPCSGRVEVKHGDTWGSICDSDFSLEAASVLCRELQCGTVVSILGGAHFGEGNGQIWAEEFQCEGHESHLSLCPVAPRPEGTCSHSRDVGVVCSRYTEIRLVNGKTPCEGRVELKTLGAWGSLCNSHWDIEDAHVLCQQLKCGVALSTPGGARFGKGNGQIWRHMFHCTGTEQHMGDCPVTALGASLCPSEQVASVICSGNQSQTLSSCNSSSLGPTRPTIPEESAVACIESGQLRLVNGGGRCAGRVEIYHEGSWGTICDDSWDLSDAHVVCRQLGCGEAINATGSAHFGEGTGPIWLDEMKCNGKESRIWQCHSHGWGQQNCRHKEDAGVICSEFMSLRLTSEASREACAGRLEVFYNGAWGTVGKSSMSETTVGVVCRQLGCADKGKINPASLDKAMSIPMWVDNVQCPKGPDTLWQCPSSPWEKRLASPSEETWITCDNKIRLQEGPTSCSGRVEIWHGGSWGTVCDDSWDLDDAQVVCQQLGCGPALKAFKEAEFGQGTGPIWLNEVKCKGNESSLWDCPARRWGHSECGHKEDAAVNCTDISVQKTPQKATTGRSSRQSSFIAVGILGVVLLAIFVALFFLTKKRRQRQRLAVSSRGENLVHQIQYREMNSCLNADDLDLMNSSENSHESADFSAAELISVSKFLPISGMEKEAILSHTEKENGNL

SEQ ID NO: 49 - protein, 354 aa, Homo sapiens
MRLAVLFSGALLGLLAAQGTGNDCPHKKSATLLPSFTVTPTVTESTGTTSHRTTKSHKTTTHRTTTTGTTSHGPTTATHNPTTTSHGNVTVHPTSNSTATSQGPSTATHSPATTSHGNATVHPTSNSTATSPGFTSSAHPEPPPPSPSPSPTSKETIGDYTWTNGSQPCVHLQAQIQIRVMYTTQGGGEAWGISVLNPNKTKVQGSCEGAHPHLLLSFPYGHLSFGFMQDLQQKVVYLSYMAVEYNVSFPHAAQWTFSAQNASLRDLQAPLGQSFSCSNSSIILSPAVHLDLLSLRLQAAQLPHTGVFGQSFSCPSDRSILLPLIIGLILLGLLALVLIAFCIIRRRPSAYQAL

SEQ ID NO: 50 - protein, 273 aa, Homo sapiens
MTFDDLKIQTVKDQPDEKSNGKKAKGLQFLYSPWWCLAAATLGVLCLGLVVTIMVLGMQLSQVSDLLTQEQANLTHQKKKLEGQISARQQAEEASQESENELKEMIETLARKLNEKSKEQMELHHQNLNLQETLKRVANCSAPCPQDWIWHGENCYLFSSGSFNWEKSQEKCLSLDAKLLKINSTADLDFIQQAISYSSFPFWMGLSRRNPSYPWLWEDGSPLMPHLFRVRGAVSQTYPSGTCAYIQRGAVYAENCILAAFSICQKKANLRAQ

SEQ ID NO: 51 - protein, 830 aa, Homo sapiens
MGLGLLLPLLLLWTRGTQGSELDPKGQHVCVASSPSAELQCCAGWRQKDQECTIPICEGPDACQKDEVCVKPGLCRCKPGFFGAHCSSRCPGQYWGPDCRESCPCHPHGQCEPATGACQCQADRWGARCEFPCACGPHGRCDPATGVCHCEPGWWSSTCRRPCQCNTAAARCEQATGACVCKPGWWGRRCSFRCNCHGSPCEQDSGRCACRPGWWGPECQQQCECVRGRCSAASGECTCPPGFRGARCELPCPAGSHGVQCAHSCGRCKHNEPCSPDTGSCESCEPGWNGTQCQQPCLPGTFGESCEQQCPHCRHGEACEPDTGHCQRCDPGWLGPRCEDPCPTGTFGEDCGSTCPTCVQGSCDTVTGDCVCSAGYWGPSCNASCPAGFHGNNCSVPCECPEGLCHPVSGSCQPGSGSRDTALIAGSLVPLLLLFLGLACCACCCWAPRSDLKDRPARDGATVSRMKLQVWGTLTSLGSTLPCRSLSSHKLPWVTVSHHDPEVPFNHSFIEPPSAGWATDDSFSSDPESGEADEVPAYCVPPQEGMVPVAQAGSSEASLAAGAFPPPEDASTPFAIPRTSSLARAKRPSVSFAEGTKFAPQSRRSSGELSSPLRKPKRLSRGAQSGPEGREAEESTGPEEAEAPESFPAAASPGDSATGHRRPPLGGRTVAEHVEAIEGSVQESSGPVTTIYMLAGKPRGSEGPVRSVFRHFGSFQKGQAEAKVKRAIPKPPRQALNRKKGSPGLASGSVGQSPNSAPKAGLPGATGPMAVRPEEAVRGLGAGTESSRRAQEPVSGCGSPEQDPQKQAEEERQEEPEYENVVPISRPPEP

(19) United States
(12) Patent Application Publication
(10) Pub. No.: US 2015/0099297 A1
(43) Pub. Date: Apr. 9, 2015

(54) CHIMERIC PROTEINS FOR TREATMENT OF DISEASES

(71) Applicant: Scaratech Medical AB, Sundbyberg (SE)

(72) Inventors: Karl Tryggvason, Djursholm (SE); Timo Pikkarainen, Rosersberg (SE); Juha Ojala, Solna (SE); Jonas Axelsson, Stockholm (SE)

(21) Appl. No.: 14/385,013

(22) PCT Filed: Mar. 12, 2013
(86) PCT No.: PCT/IB2013/000845

§ 371 Date: Sep. 12, 2014

Related U.S. Application Data
(60) Provisional application No. 61/609,523, filed on Mar. 12, 2012.

(51) Int. Cl. C07K 14/705; A61M 1/36; B01D 15/38

(52) U.S. Cl. CPC C07K 14/705; B01D 15/3823; A61M 1/36; C07K 2319/20; C07K 2319/21; C07K 2319/22; C07K 2319/30; C07K 2319/02
USPC 435/328; 530/387.3; 536/23.4; 435/320.1; 435/269

(57) ABSTRACT

A chimeric protein is made from the combination of (i) a pathogen recognition module derived from a scavenger receptor and (ii) an anchor domain from a different scavenger receptor. The chimeric protein binds to specific pathogens and is useful in various treatments.

This application claims priority to U.S. Provisional Patent Application Ser. No. 61/609,523, filed on Mar. 12, 2012. The entirety of that disclosure is hereby fully incorporated by reference herein.

### BACKGROUND

A sequence listing is being submitted herein as an ASCII text file with the name “SCAR200003_ST25.txt”, created on Mar. 12, 2013, with a file size of 123,236 bytes. The material in this text file is hereby fully incorporated by reference herein.

The present disclosure relates to methods, materials, compositions, applications, and therapies that involve the creation and use of recombinant chimeric proteins, which may be referred to herein as hybrid scavenger receptors.

Sepsis is commonly defined as the combination of pathologic infection and physiological changes known collectively as the systemic inflammatory response syndrome (SIRS). Septicemia is a related term that refers to the presence of pathogenic organisms in the bloodstream, leading to sepsis. These two terms are generally used interchangeably herein.

Sepsis is associated with a high mortality rate, reported to be 20% to 50% in severely affected patients despite treatment with antibiotics. Sepsis is the second leading cause of death among patients in non-coronary intensive care units (“ICU”) and the 10th leading cause of death overall in the United States, with figures for Europe likely to be similar. Furthermore, sepsis is a leading cause of death in young individuals and a life-long reduction in quality of life is seen amongst those who survive. Current sepsis therapy consists mainly of broad-spectrum antibiotics and organ support, and is often ineffective. The efficacy of therapy is projected to decrease with increased antibiotic resistance amongst pathogens.

There has been a substantial increase in the incidence of sepsis during the past 22 years with an increasing number of deaths occurring despite a decline in overall in-hospital mortality. Using ICD-9 codes, a profile of sepsis in the US from hospital discharge records has been created, and it is estimated that more than 751,000 cases of severe sepsis occur yearly, accounting for 2.1% to 4.3% of hospitalizations and 11% of all admissions to the ICU. It has also been estimated that the incidence of sepsis in the US has increased significantly over the past two decades. Possible reasons for this increase include the increased use of invasive procedures; immunosuppressive drugs, chemotherapy, and transplantation; the emergence of the epidemic of human immunodeficiency virus (HIV) infection; and increasing microbial resistance. The relative frequency of specific causative organisms has shifted over time, with a recent pre-eminence of gram-positive organisms. Alarmingly, the occurrence of organ failure due to sepsis has increased over time and is a major contributor to mortality. The same is likely to have occurred in Europe.

### BRIEF DESCRIPTION

Disclosed herein are chimeric proteins which can be used to bind to pathogens. Briefly, each protein contains a pathogen recognition module that is derived from a mammalian scavenger receptor. Each protein also contains an anchor domain for fixing the chimeric protein in place.

Disclosed in various embodiments herein is a recombinant chimeric protein comprising: a pathogen recognition module from a mammalian scavenger receptor; and an anchor domain.

The protein may further comprise a linker between the pathogen recognition module and the anchor domain. The linker can be a sequence of about 2 to about 5 amino acids. The linker may include at least two different amino acids. In some embodiments, these two different amino acids are selected from the group consisting of alanine, histidine, and glycine.

The protein may further comprise an affinity tag. The affinity tag can be selected from the group consisting of hemagglutinin, V5, Myc, T7, FLAG, HSV, VSV-G, 6-His, biotin/streptavidin, and STREP.

The pathogen recognition module may be a pathogen-binding domain from the family of mammalian cell-surface receptors such as for example SCARA3, CD36, CD163, CD68, LOX-1, SREC-1 or SCARA5. These scavenger receptors contain pathogen-binding domains such as a conserved SRCR domain, a C-type lectin-like domain (CTLD), or a collagenous sequence.

The anchor domain can be an Fc region of an immunoglobulin. Alternatively, the anchor domain can be an alpha-helical coiled coil domain from a mammalian scavenger receptor. The anchor domain may also be an affinity tag. In particular embodiments of the present disclosure, the mammalian scavenger receptor that the pathogen recognition module is derived from is different from the mammalian scavenger receptor that the anchor domain is derived from.

The protein can further comprise a signal peptide.

Also disclosed herein are: a nucleic acid sequence encoding for the chimeric receptor protein; a vector comprising such a nucleic acid sequence; and a host cell including such a vector.

Disclosed herein also is a pharmaceutical composition comprising a chimeric protein and a pharmaceutically acceptable carrier; wherein the chimeric protein comprises: a pathogen recognition module from a scavenger receptor; and an anchor domain.

Also disclosed herein is an affinity medium comprising a solid phase and a chimeric protein immobilized upon the solid phase; wherein the chimeric protein comprises: a pathogen recognition module from a scavenger receptor; an optional linker; and an anchor domain, the anchor domain being attached to the solid phase.

Furthermore, the present disclosure describes methods to optionally regenerate the disclosed affinity medium, by treating the medium so that the bound pathogen or pathogen-derived molecules are dislodged, while the couplings within the solid phase and between the chimeric protein and the solid phase remain undisturbed.

The present disclosure also describes a method for treating a disease associated with a pathogen or a pathogen-derived molecule, comprising: receiving an affinity medium comprising a solid phase and a chimeric protein immobilized upon the solid phase, the chimeric protein comprising (i) a pathogen recognition module from a scavenger receptor and (ii) an anchor domain; and contacting a biological sample with the affinity medium to bind the pathogen or pathogen-derived molecule to the affinity medium.

Also described herein is a method for determining the efficacy of a test affinity medium for a pathogenic agent, comprising: receiving a biological sample containing the pathogenic agent; contacting the biological sample with a pathogen recognition module of the test affinity medium; and detecting the amount of binding between the pathogenic agent and the pathogen recognition module. This method may further comprise comparing the amount of binding between the pathogenic agent and the pathogen recognition module of the test affinity medium to a reference.

Disclosed in embodiments here is a medicament for the prevention or treatment of a disease associated with a pathogen or a pathogen-derived molecule, comprising a chimeric protein in a carrier suitable for topical application.

Also disclosed is a recombinant chimeric protein, consisting of: a signal peptide; at least one affinity tag; an optional linker; a pathogen recognition module from a mammalian scavenger receptor; and an anchor domain.

Also disclosed herein is a method for diagnosing whether a patient has a disease associated with a pathogen or a pathogen-derived molecule, comprising: receiving an affinity medium comprising a solid phase and a chimeric protein immobilized in a fixed space upon the solid phase, the chimeric protein comprising (i) a pathogen recognition module from a scavenger receptor and (ii) an anchor domain; receiving a biological sample obtained from the patient; contacting the biological sample with the affinity medium to bind any pathogen or pathogen-derived molecule to the fixed space; and examining the fixed space for the presence of any bound pathogen or pathogen-derived molecule. The examining can be performed by hybridizing any bound pathogen or pathogen-derived molecule with a labeled probe.

These and other non-limiting characteristics are more particularly described below.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following is a brief description of the drawings, which are presented for the purposes of illustrating the exemplary embodiments disclosed herein and not for the purposes of limiting the same.

FIG. 1A and FIG. 1B are diagrams showing the structural similarities and differences between different classes of scavenger receptor proteins. The diagrams are modified from Platt, N. et al. Trends Cell Biol. Volume 8, Issue 9, 1 Sep. 1998, Pages 365-372. The diagrams do not accurately show the relative sizes of these proteins.

FIG. 2 is a diagram showing one possible use of the chimeric proteins of the present disclosure.

FIG. 3 is a microphotograph used for a densitometry analysis showing protein bound to a solid phase (here, beads).

FIG. 4 is a graph showing a Bradford assay standard curve used for analysis of the amount of protein bound to a solid phase.

FIG. 5 is a set of graphs showing the results when four different bacteria are exposed to an affinity medium having a first chimeric protein. The graphs show the amount of viable bacteria over a course of consecutive fractions washed from the affinity medium, and a count of total surviving bacteria over the course of the experiment.

FIG. 6 is a set of graphs showing the results when four different bacteria are exposed to an affinity medium having a second chimeric protein. The graphs show the amount of viable bacteria over a course of consecutive fractions washed from the affinity medium, and a count of total surviving bacteria over the course of the experiment.

FIG. 7 is a set of graphs showing the results when extended-spectrum β-lactamase producing E. coli (ESBL) bacteria are exposed to an affinity medium having a third chimeric protein. The graphs show the amount of viable bacteria over a course of consecutive fractions washed from the affinity medium, and a count of surviving bacteria remaining in the column at the end of the experiment.

FIG. 8 is a set of graphs showing the results when extended-spectrum β-lactamase producing E. coli (ESBL) bacteria are exposed to an affinity medium having a fourth chimeric protein. The graphs show the amount of viable bacteria over a course of consecutive fractions washed from the affinity medium, and a count of surviving bacteria remaining in the column at the end of the experiment. The mean and standard deviation derived from three separate experiments are shown.

FIG. 9A and FIG. 9B are graphs showing TNF production in peripheral blood mononuclear cells (PBMCs) exposed to each column fraction derived from Example 1 (i.e. the first chimeric protein).

FIG. 10A and FIG. 10B are graphs showing TNF production in peripheral blood mononuclear cells (PBMCs) exposed to each column fraction derived from Example 2 (i.e. the second chimeric protein).

FIG. 11 is a set of pictures showing the binding of heat-inactivated E. coli and acetylated low-density lipoprotein (acLDL) to two different chimeric proteins, intact SCARA5 and a control. Expression was confirmed using an anti-SCARA5 antibody.

FIG. 12 is a set of pictures showing the internalization of bacterial lipopolysaccharide (LPS) by SCARA5 transfected cells compared to control Crb2 cells. Expression of SCARA5 and Crb2 was confirmed using an anti-SCARA5 and anti-Crb2 antibodies.

FIG. 13 is a first set of graphs showing TNF production induced in peripheral blood mononuclear cells (PBMCs) from donors and exposed to column flow-through obtained by passing pathogens mixed in human serum through a column containing beads coupled to different chimeric receptor proteins. The control group consisted of beads without chimeric receptors bound.

FIG. 14 is a second set of graphs like FIG. 13, but with different pathogens.

FIG. 15 shows the results of dot blots performed using the same equipment as in FIG. 13 and FIG. 14 but using different wash solutions to elute the bound pathogen from the control beads and beads loaded with chimeric receptors (SEQ ID NO: 35, SEQ ID NO: 43, and SEQ ID NO: 41). In this case, biotinylated MRSA is shown in the material applied to the column (loading) and after passage and washing (detection of biotin) using an anti-biotin HRP-conjugated antibody. Wash solution 1 does not elute the bound MRSA in any of the environmental conditions, but Wash solution 2 does so for beads loaded with SEQ ID NO: 41 and SEQ ID NO: 43. After eluting everything from the beads with wash solution 3, no bound bacteria fragments (biotin) is detected in the control beads or beads loaded with SEQ ID NO: 35.

FIG. 16 is a set of five immunohistochemistry (IHC) pictures of proteins tagged with one set of affinity tags, to be compared with FIG. 17. The amount of protein present is high.

FIG. 17 is a set of five immunohistochemistry (IHC) pictures of proteins tagged with another set of affinity tags, to be compared with FIG. 16. The amount of protein present is low.

FIG. 18 is a set of Western blots showing the affinity of two chimeric receptors (expressed from SEQ ID NO: 43 and SEQ ID NO: 41) for three species of pathogenic bacteria or naked matrix. The left pane shows the presence of each receptor protein in the initial solution. The right pane shows that the proteins are still attached to the beads after the addition of various bacteria and running the wash protocol. The same is true for His-tagged proteins attached to TALON beads as well as for AviTagged, biotinylated proteins attached to streptavidin-coated agarose. It may be seen that for gram-positive bacteria (but not for gram-negative E. coli or His-tagged protein) the concentration of chimeric receptor dropped following admixture—indicating that binding between pathogen molecules/agarose and chimeric receptors has occurred, such that some protein is no longer in the solution.

### DETAILED DESCRIPTION

A more complete understanding of the compositions and methods disclosed herein can be obtained by reference to the accompanying drawings. These figures are merely schematic representations based on convenience and the ease of demonstrating the present disclosure, and are, therefore, not intended to define or limit the scope of the exemplary embodiments.

Although specific terms are used in the following description for the sake of clarity, these terms are intended to refer only to the particular structure of the embodiments selected for illustration in the drawings, and are not intended to define or limit the scope of the disclosure. In the drawings and the following description below, it is to be understood that like numeric designations refer to components of like function.

All publications, patents, and patent applications discussed herein are hereby incorporated by reference in their entirety.

The present disclosure relates to chimeric proteins that are based on mammalian scavenger receptor proteins (for the purpose of this disclosure abbreviated to scavenger receptors, or simply SRs). There are about 30 human receptor proteins (both soluble and membrane-bound) that have one or more occurrences of an ancient and highly conserved protein module—the scavenger receptor cysteine-rich (SRCR) domain. SRCR domains are approximately 90 to 110 amino acids long and have a high and well defined cysteine content. Depending on the characteristics of their SRCR domains, two types of SR members are reported: those with type A domains (SCARAs; encoded by at least two exons and containing six cysteine residues), and those with type B domains (encoded by a single exon and containing eight cysteine residues). Some exceptions occur, including one SCARA which presents truncated SRCR domains containing four cysteines. Likewise, isolated domains containing six cysteines are found among group B members, as is the case with CD5, CD163, WC1, and MC16. Moreover, Spa/AIM, WC1/T19 and CRP-ductin (mouse DMBT1) possess individual domains containing seven cysteines. However, the simultaneous presence of type A and B domains on the same scavenger receptor has never been reported. Sequence analysis has revealed that although SRCR domains share different levels of homology, the relative position of cysteines is well conserved within the domain, as is the pattern of disulfide bonds. Thus, sequence analysis revealed that cysteines C1 and C4 form a disulfide bond, because they are always present in group B but not group A. Proteolytic analysis showed that the other cysteine pairs forming disulfide bonds are C2-C7, C3-C8, and C5-C6. These results have been confirmed by structural analysis of crystallized individual protein domains.

Besides the discussed SRCR domain, any of the SRs are characterized by their location on the surface of subpopulations of immunocompetent cells of hematopoietic lineage. In addition, varying numbers and degrees of SR expression can be found in almost all other cell types.—from where they are capable of binding a large number of endogenous ligands. These include acetylated or oxidized low-density cholesterol (LDL) as well as normal high-density cholesterol (HDL), hemoglobin, iron and carbohydrates. SRs also bind both gram-positive and gram-negative bacteria.

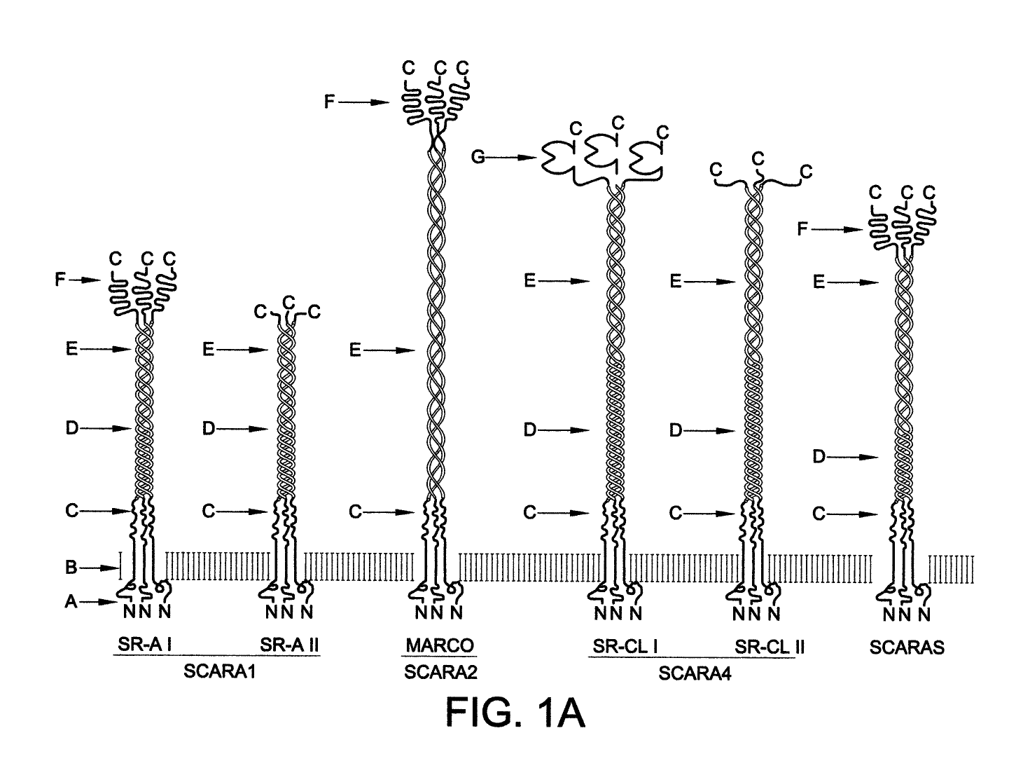

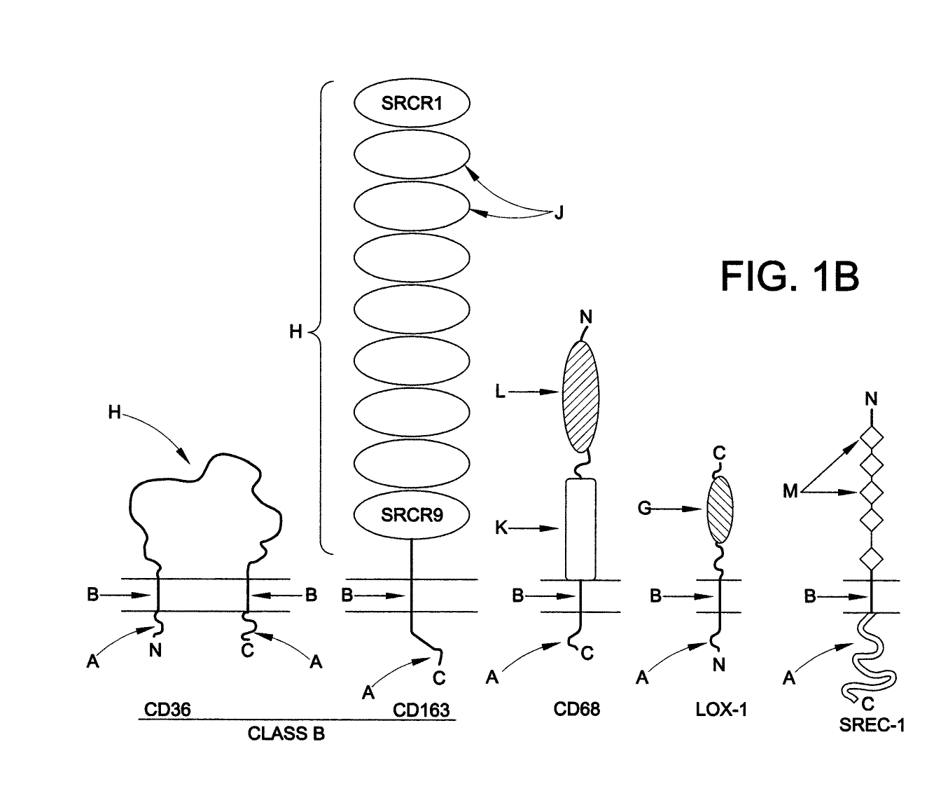

FIG. 1 illustrates the structural features of different classes of scavenger receptors (SRs).

Class A SRs are trimeric glycoproteins, of which there are several subtypes: SR-A type I and II (SCARA1), MARCO (SCARA2), SR-CL type I and II (SCARA4), and SCARA5. A SCARA3 subtype is not illustrated here. The trimeric molecule is built up of blocks including a cytoplasmic domain A, a transmembrane domain B, a spacer domain C, an alpha-helical coiled-coil domain D, a collagenous triple helix domain E, a cysteine-rich domain F, or a C-type lectin-like domain G. The type I SR-A receptor has six domains, while the type II receptor does not have a cysteine-rich domain F. The MARCO receptor does not have an alpha-helical coiled-coil domain D, and has a relatively long collagenous triple helix domain E. In the SR-CL type I receptor, the cysteine-rich domain F is replaced by the C-type lectin-like domain G. The SCARA5 receptor is similar to the SR-A type I receptor with six domains. In Class A SRs, the collagenous domain E, the cysteine-rich domain F, or C-type lectin-like domain G include binding sites for acetylated low-density lipoprotein or bacteria and other ligands. It should also be noted that in Class A SRs, the N-terminal end is in the cytoplasm.

Class B SRs include, for example, CD36 and CD163. The CD36 protein adopts a ditopic configuration, with an extracellular domain H flanked by two transmembrane domains B and two cytoplasmic domains A. The extracellular domain contains a hydrophobic domain, a proline rich region, and several binding sites. The CD163 protein has a cytoplasmic domain A at its C-terminal end, a transmembrane domain B, and an extracellular domain which consists exclusively of nine class B SRCR domains J, numbered with SRCR1 at the N-terminal end and SRCR9 near the C-terminal end. The class B SRCR domains have four disulfide bridges, whereas class A SRCR domains have three disulfide bridges.

Class D SRs include, for example, CD68 (also known as macrosialin). The C-terminal end of CD68 is in the cytoplasm. CD68 includes a cytoplasmic domain A, a transmembrane domain B, a lysosomal-associated membrane protein (LAMP)-like domain K, and a mucin-like domain L.

The only current Class E SR is LOX-1. The N-terminal end of LOX-1 is in the cytoplasm. LOX-1 includes a cytoplasmic domain A, a transmembrane domain B, and a C-type lectin domain G. The C-type lectin-like domain (CTLD) containing lectins are Ca²⁺-dependent carbohydrate-binding proteins. The structural superfamily also contains Ca²⁺-independent lectins and proteins with a lectin-like domain but unable to bind carbohydrates. However, the CTLD fold can be recognized from its four (in some cases six) conserved cysteine residues. Proteins with CTLDs are not obligatory SRs, but also include some soluble proteins e.g. collectins such as mannose binding lectin (MBL) and surfactant proteins (SP-A and SP-D). However, CTLDs are known to be present in the SRs mannose receptor, LOX-1, and SCARA4.

The only current Class F SR is SREC-1. The C-terminal end of SREC-1 is in the cytoplasm. SREC-1 includes a cytoplasmic domain A, a transmembrane domain B, and an extracellular domain having five EGF-like domains M.

The amino acid sequence for the SCARA3 protein is SEQ ID NO: 46. The structure of SCARA3 is similar to SCARA1 splice variant II and contains a N-terminal cytoplasmic domain, a transmembrane domain, a spacer domain, a coiled-coil domain, and a collagenous domain. The C-terminal collagenous domain amino acid residues 440-543 and 513-572 include the binding sites for ligands such as cleavage and polyadenylation-specific factor 3 (CPSF3) and X-linked inhibitor of apoptosis protein (XIAP).

The amino acid sequence for the CD36 protein is SEQ ID NO: 47. The CD36 protein includes OxLDL binding sites between amino acids 155-183, amino acids 28-93, and amino acids 120-155. Other binding sites are also present between amino acids 93-120. The domain between amino acids 139-184, amino acids 146-164, and amino acids 145-171 have also been shown to mediate binding with PfEMP-1.

The amino acid sequence for the CD163 protein is SEQ ID NO: 48. Each separate SRCR domain J is a binding domain, as are combinations of adjacent SRCR domains. The SRCR1 domain is encoded by amino acids 1-148. The SRCR2 domain is encoded by amino acids 149-255. The SRCR3 domain is encoded by amino acids 256-362. The SRCR4 domain is encoded by amino acids 363-469.

The amino acid sequence for the CD68 protein is SEQ ID NO: 49. Both the LAMP-like domain K and the mucin-like domain L are binding sites.

The amino acid sequence for the LOX-1 protein is SEQ ID NO: 50. The C-type lectin domain G is a binding site.

The amino acid sequence for the SREC-1 protein is SEQ ID NO: 51. Each EGF-like domain M is a binding site. An EGF-like domain contains 30-40 amino acid residues and 2-3 disulfide bonds, and has the structure of two β-sheets.

Unlike synthetic antibiotics, the SRs have co-evolved with human pathogens, and constitute an important part of the innate immune defense by acting as pattern-recognition receptors, in particular against bacterial pathogens. Several SRs are expressed on macrophages and dendritic cells, where they act as phagocytic receptors mediating binding and uptake of pathogenic microbes including gram-positive and gram-negative bacteria, intracellular bacteria and viral RNA. SRs also act as co-receptors to Toll-like receptors (TLR), modulating the inflammatory response to TLR agonists. SRs have commonly been reported to bind lipopolysaccharide (LPS) and lipoteichoic acid (LTA), which are present on the surface of gram-negative and gram-positive bacteria, respectively. Recent data indicate that bacterial surface proteins also act as important target molecules for SRs. Moreover, intracellular pathogens, including viruses and Plasmodium falciparum, use SRs to gain entry into host cells, thus making them obligatory SR-binding. Unlike non-specific bacterial ligands such as heparin sulphate, SRs exhibit strong binding to target pathogens in addition to their recognition of multiple pathogenic ligands, likely making them good candidates for use in extracorporeal removal of bacteria from blood.

The present disclosure uses the evolutionarily conserved SR system as a therapy and diagnostic for pathogenic agents. A number of recombinant cell lines expressing examples of relevant natural mammalian and chimeric SRs have been established. Technologies to end-point attach these SRs to a solid phase, such as a bead matrix, flat surface or machine, have also been developed. Thus, it is feasible to create an affinity medium that can be used for therapies such as extracorporeal blood treatments.

Disclosed herein are recombinant chimeric proteins, which can be used to bind to pathogens. Briefly, each protein contains a pathogen recognition module (PRM) that is derived from a scavenger receptor. Each protein also contains an anchor domain for fixing the chimeric protein in place upon a support. These proteins may be referred to as chimeric scavenger receptors (cSR).

The pathogen recognition module may be derived from the binding domain of a mammalian scavenger receptor, and may thus be tailored to a specific group of pathogens or pathogen-derived molecules. For example, the binding domain can be that of class A scavenger receptor member 2 (SCARA2), known to bind Clostridium sordelli, or that of class B scavenger receptor member LOX-1, known to bind S. aureus. The binding domain binds to a specific atom or molecule. Referring to the parts of a scavenger receptor illustrated in FIG. 1A and FIG. 1B, any of the binding domains described above may be considered a pathogen recognition module. In particular embodiments, the pathogen recognition module is located at the C-terminal end of the chimeric receptor protein.

The anchor domain is attached to the pathogen recognition module, either directly or indirectly, and is used to end-point immobilize the protein to a solid phase or support. The anchor domain may also allow for limited special reconfiguration of the pathogen recognition module or allow multiple PRMs to bind to a single target molecule. The anchor domain, in some embodiments, can be an Fc region of an immunoglobulin. In other particular embodiments, the anchor domain is the coiled-coil domain from a mammalian SR (letter D in FIG. 1A). Alternatively, the anchor domain can be an affinity tag (which are further described herein). The anchor domain is typically at one end of the chimeric protein.

In specific contemplated embodiments, the pathogen recognition module and the anchor domain are obtained from different Class A scavenger receptor proteins.

If desired, a linker may be present between the pathogen recognition module and the anchor domain. This linker increases the distance between the PRM and the anchor domain of the cSR and thus allows for different pathogen specificity and binding strength. The linker also allows for correct protein folding and secretion during manufacturing. In embodiments, the linker may be a sequence having a length of from about 2 to about 15 amino acids. The linker may include at least two different amino acids. In some embodiments, the linker contains at least two different amino acids selected from the group consisting of alanine (A), histidine (H), and glycine (G). Five non-limiting examples of useful linker sequences are SVEA (SEQ ID NO: 1), DMDF (SEQ ID NO: 2), GAAGG (SEQ ID NO: 11), AAAGG (SEQ ID NO: 12), and HHK (SEQ ID NO: 14).

Additionally, the cSR may contain one or more affinity tags, either in tandem with the protein (i.e. at one end thereof) or at a location within the protein (e.g. between the PRM and the anchor domain). An affinity tag is a sequence that generally permits the chimeric protein to be purified using an affinity technique and/or to be attached to an affinity surface with a known orientation. Several different kinds of affinity tags are known in the art. In particular embodiments, the affinity tag is selected from the group consisting of hemagglutinin, AviTag™, V5, Myc, T7, FLAG, HSV, VSV-G, His, biotin, or STREP. The sequence for the hemagglutinin tag is provided as SEQ ID NO: 22. The sequence for AviTag™ is provided as SEQ ID NO: 32. The sequence for the V5 tag is provided as SEQ ID NO: 23. The sequence for the Myc tag is provided as SEQ ID NO: 24. The sequence for the FLAG tag is provided as SEQ ID NO: 25. The sequence for the HSV tag is provided as SEQ ID NO: 26. The sequence for the VSV-G tag is provided as SEQ ID NO: 27. The sequence for the His tag is provided as SEQ ID NO: 28. The sequence for the T7 tag is provided as SEQ ID NO: 29. The sequence for the biotin tag is provided as SEQ ID NO: 30. The sequence for the STREP tag is provided as SEQ ID NO: 31. When more than one affinity tag is used in tandem, a linker sequence may be present to join the affinity tag to the pathogen recognition module and anchor domain. As an example, the linker between the affinity tags His (SEQ ID NO: 28) and biotin (SEQ ID NO: 30) be SEQ ID NO: 13. It should be noted that the anchor domain can be an affinity tag.

The chimeric protein may also include a signal peptide that is used to direct the secretion and/or transport of the protein in the synthesizing cell. The signal peptide may be located at one end of the chimeric protein. Two examples of such signal peptides are peptides from the TIMP2 and BM-40 proteins (SEQ ID NO: 15 and SEQ ID NO: 16, respectively).

It should be noted that as the pathogen recognition module and the anchor domain of the cSR disclosed herein are each derived from different mammalian SRs, the present disclosure does not intend to encompass natural SRs. This is indicated by the use of the terms “recombinant” and “chimeric”, which refer to the fact that the protein is artificially created and may include functional units and fragments of different proteins. The SRs as depicted in FIG. 1A and FIG. 1B are specifically excluded from the scope of the present disclosure.

Also disclosed herein are nucleic acid sequences that encode the chimeric proteins described above. The nucleic acid sequence may be DNA or RNA. As is known in the art, the sequence of amino acids in a protein is defined by the nucleic acid sequence, which is transcribed and/or translated to produce the protein. Each triplet of nucleotides in the nucleic acid sequence designates a specific amino acid, as is known, and so appropriate nucleic acid sequences are known if the protein is known, and vice versa.

The chimeric proteins of the present disclosure can be produced by inserting the cSR nucleotide sequence into an expression vector, which is subsequently transfected into a host cell. Exemplary vectors include plasmids and viruses. The chimeric proteins are then produced by the host cell and can subsequently be isolated. These methods are well known to those skilled in the art.

The chimeric proteins may be used to make a pharmaceutical composition comprising a chimeric protein and a pharmaceutically acceptable carrier. The carriers can be injectable carriers, topical carriers, transdermal carriers, and the like. The preparation may advantageously be in a form for topical administration, such as an ointment, gel, cream, spray, dispersion, suspension or paste. The preparations may further advantageously include preservatives, antibacterials, antifungals, antioxidants, osmotic agents, and similar materials in composition and quantity as is conventional. Suitable solutions for use in accordance with the disclosure are sterile, are not harmful for the proposed application, and may be subjected to conventional pharmaceutical operations such as sterilization and/or may contain conventional adjuvants, such as preservatives, stabilizers, wetting agents, emulsifiers, buffers etc. For assistance in formulating the compositions of the present disclosure, one may refer to Remington's Pharmaceutical Sciences, 15th Ed., Mack Publishing Co., Easton, Pa. (1975).

The chimeric protein may be immobilized upon a support or solid phase to make an affinity medium, which can also be referred to herein as an anti-pathogen immunoadhesin (API). The anchor domain of the chimeric protein is attached to the solid phase, permitting the pathogen recognition module to interact with its specific pathogen or pathogen-derived molecule. The solid phase may, for example, be in the form of a bead, a sheet or strip, a pleated sheet, etc.

Methods for using the chimeric protein are also contemplated. One method for preventing or treating a disease associated with a pathogen or a pathogen-derived molecule involves an affinity medium as described above, which constitutes a solid phase and one or more cSRs attached thereto. Each chimeric protein (cSR) comprises a pathogen recognition module and an anchor domain, as well as optionally one or more linkers. A biological sample (such as blood) is removed from the diseased individual and contacted with the affinity medium to bind any pathogen or pathogen-derived molecule contained in said sample. Following this, the sample is returned to the diseased individual minus the bound particles. This permits the pathogen/pathogen-derived molecule which is targeted by the chimeric protein to be removed from the biological sample. Optionally, the affinity medium may then be regenerated by eluting bound molecules as disclosed herein, and subsequently reusing the medium for the same or a different purpose.

The chimeric protein can also be used as a medicament for the prevention or treatment of a disease associated with a pathogen or a pathogen-derived molecule. In such an embodiment, one or more cSRs is used with or without an affinity medium in a pharmaceutical composition as previously described. The affinity medium could furthermore in itself have direct or indirect effects on the target pathogen, such that the cSR brings the solid phase to specific organisms or molecules. The pharmaceutical composition is applied topically or as dictated by the localization of the pathogen or pathogen derived molecules. For example, in one embodiment of the present disclosure, a cream containing selected cSRs with a high affinity for human immunodeficiency virus (HIV) may be applied to the genital area to inactivate HIV and/or to prevent its invasion of host cells. In another embodiment of the disclosure, a gel containing one or more cSRs known to bind bacteria may be incorporated in a wound dressing to immobilize and/or destroy bacteria interfering with wound healing. The pharmaceutical composition can be used for preventative or therapeutic applications.

Methods for determining the efficacy of a test affinity medium for a pathogenic agent are also contemplated. A biological sample containing the pathogenic agent is received. The biological sample is then contacted with a test affinity medium that contains a pathogen recognition module. The amount of binding between the pathogenic agent and the pathogen recognition module is detected/measured, for example by using immunohistochemical means like colorimetry, linked secondary antibodies, surface plasmon resonance imaging, or high-pressure liquid chromatography (HPLC). The amount of binding between the pathogenic agent and the pathogen recognition module of the test affinity medium can be compared to a reference. Binding of ligands to the affinity medium indicates that the affinity medium is likely to be effective in immobilizing/targeting the pathogenic agent. Alternatively, the amount of viable pathogens remaining in a sample or the ability of said sample to induce reactions in tissues ex vivo (eg. isolated PBMCs) may be determined following passage of the sample over the affinity media. A lower amount of viable pathogens or a more benign reaction indicates that the affinity medium may be effective in reducing the amount of the pathogen or pathogenic agents. This type of test can also be done using a test cell that expresses the pathogen recognition module. The test cell would also contain a signaling means that operates through the pathogen recognition module. The level of signaling would then indicate the degree of binding. A decrease in signaling would indicate that the specific cSR expressed by the test cell is a good candidate for immobilizing the pathogenic agent.

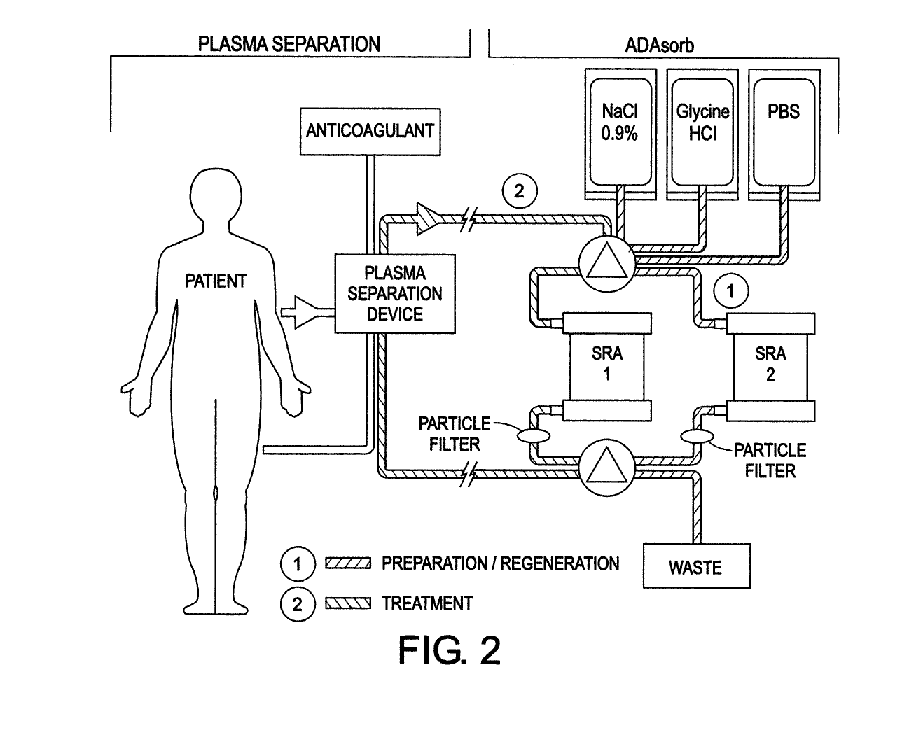

An exemplary use of the chimeric proteins is shown in FIG. 2. Here, the chimeric proteins are bound to a solid phase and used as a filter, in the columns labeled SRA 1 and SRA 2. A plasma separation device, for example a size-exclusion filter, takes blood from a patient and separates the blood to obtain plasma containing the pathogen. The plasma is then passed via an extracorporeal circulation pump to either the SRA 1 or the SRA 2 filter, where the pathogen is removed from the plasma. The plasma is then returned to the plasma separation device and back into the patient. A column switcher device is present. This permits one of the filters to be regenerated while the other filter is cleaning the plasma. A filter can be cleaned, for example, by elution with a low pH Glycine-HCl mixture followed by washing with PBS. The input to each filter is either (i) plasma or (ii) cleaning solution. The output from each filter is either (a) cleansed plasma or (ii) waste solution. Anticoagulant is optional, but is shown here. Fractionated heparin, citrate, and/or other such anticoagulants and methods are well known to those of ordinary skill in the art.

Aspects of the present disclosure may be further understood by referring to the following examples. The examples are illustrative, and are not intended to be limiting embodiments thereof.

### EXAMPLES

### Example 1

### Example 1. MARCO PRM with Partial MARCO Anchor

A construct encoding the cSR truncated soluble MARCO (SEQ ID NO: 7) was generated by established molecular biological methods, and contained the following elements in the pcDNA3.1/Zeo(-) mammalian expression vector (Invitrogen):

1) a secretion signal peptide from the BM-40 protein (SEQ ID NO: 16);

2) an 8-histidine-long tag for protein purification and linker (SEQ ID NO: 28 and SEQ ID NO: 14);

3) the extracellular part of mouse MARCO residues 75-518 (that correspond to nucleotides 223-1557) with deletion of residues 300-419 (nucleotides 897-1257) (SEQ ID NO: 21). This form of MARCO lacks the last 40 Gly-X-Y repeats of the 89-repeat-long collagenous domain of mouse MARCO, and as a transmembrane protein has shown to be a strong binder of the prototypic scavenger receptor ligands, heat-killed E. coli and acetylated LDL (low density lipoprotein).

An analogous cSR truncated soluble human MARCO sequence is presented as SEQ ID NO: 8. The human MARCO portion of this analogous sequence is presented as SEQ ID NO: 32.

### Example 2

### Example 2. SR-A PRM with SCARA5 Anchor

A construct encoding the cSR soluble mouse SCARA5 with the mouse SR-A1 SRCR (scavenger receptor cysteine-rich) domain (SEQ ID NO: 9) was also cloned into the pcDNA3.1/Zeo(-) vector, and contains the following elements:

1) a secretion signal peptide from the TIMP2 protein (SEQ ID NO: 15);

2) a 6-histidine-long tag for protein purification and linker (SEQ ID NO: 28 and SEQ ID NO: 13);

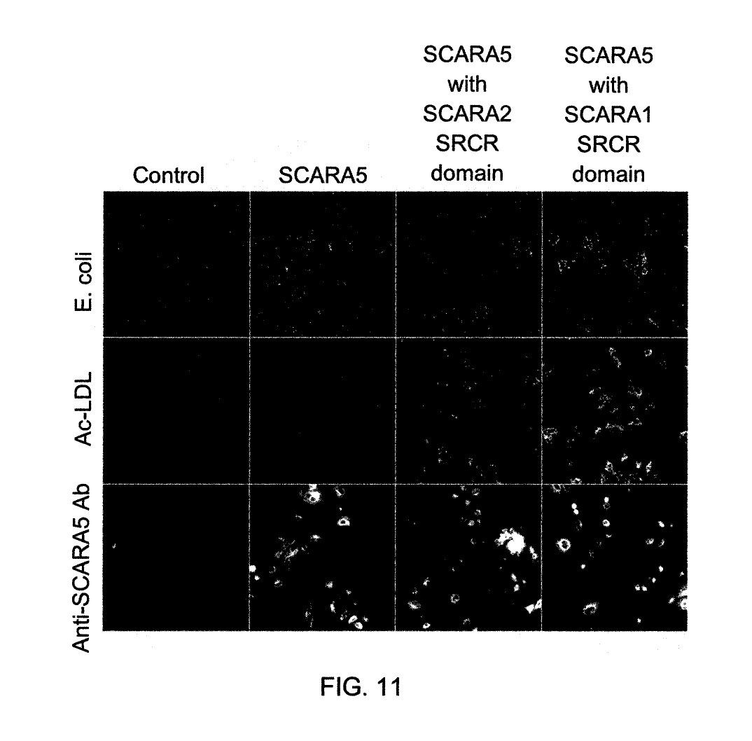

3) the extracellular part of mouse SCARA5, except that the SRCR domain of the protein was replaced with the SRCR domain from mouse SR-A In other words, this chimeric version contained residues 83-380 from SCARA5 (nucleotides 247-1140) followed by the SRCR domain from SR-A (residues 345-454 that correspond to nucleotides 1035-1362) (SEQ ID NO: 33). As a transmembrane protein, this form of SCARA5 strongly binds heat-killed E. coli and acetylated LDL (stronger than intact SCARA5), as discussed further herein with reference to FIG. 11.

An analogous cSR truncated soluble human SCARA5 sequence is presented as SEQ ID NO: 10. The human SCARA5 portion of this analogous sequence is presented as SEQ ID NO: 34.

### Example 3

### Example 3. SCARA5 PRM with Extracellular Part of SCARA5

A construct encoding the cSR soluble mouse SCARA5 (SEQ ID NO: 5) was generated by established molecular biology methods, and contained the following elements in the pcDNA3.1/Zeo(-) mammalian expression vector (Invitrogen):

1) a secretion signal peptide from the TIMP2 protein (SEQ ID NO: 15);

2) a 6-histidine-long tag for protein purification and a linker (SEQ ID NO: 28 and SEQ ID NO: 13);

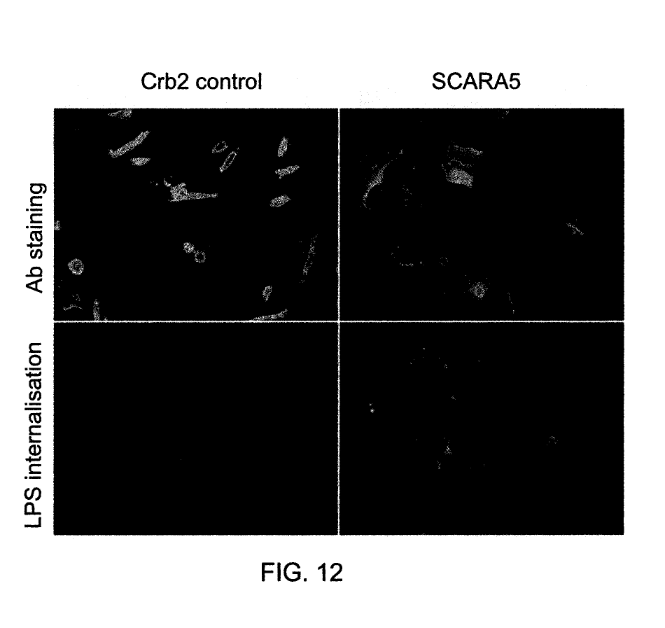

3) the extracellular part of mouse SCARA5 residues 83-491 (corresponding to the nucleotides 247-1476) (SEQ ID NO: 19). As depicted in FIG. 11 and FIG. 12, as a transmembrane protein, SCARA5 is able to bind heat killed E. coli and internalize LPS.

An analogous cSR soluble human SCARA5 sequence is presented as SEQ ID NO: 3. The human SCARA5 sequence is presented as SEQ ID NO: 17.

### Example 4

### Example 4. SCARA5 PRM with a Truncated Extracellular Part of SCARA5

A construct encoding the cSR soluble, truncated mouse SCARA5 (SEQ ID NO: 6) was generated by established molecular biology methods, and contained the following elements in the pcDNA3.1/Zeo(-) mammalian expression vector (Invitrogen):

1) a secretion signal peptide from the TIMP2 protein (SEQ ID NO: 15);

2) a 6-histidine-long tag for protein purification and a linker (SEQ ID NO: 28 and SEQ ID NO: 13);

3) the truncated extracellular part of mouse SCARA5 residues 341-491 (corresponding to the nucleotides 1021-1476) (SEQ ID NO: 20). As depicted in FIG. 11 and FIG. 12, as a transmembrane protein, SCARA5 is able to bind heat killed E. coli and internalize LPS.

An analogous cSR soluble human SCARA5 sequence is presented as SEQ ID NO: 4. The human SCARA5 sequence is presented as SEQ ID NO: 18.

### Example 5

### Example 5. Expression of Used cSRs

Plasmids were transfected into 293/EBNA cells (Invitrogen) with the Ca-phosphate transfection method. One day after transfection, cells were seeded at different densities on 10-cm tissue culture plates, and selection with zeocin (500 μg/ml) was started next day. After 2 weeks, clones were picked with cloning cylinders, and expanded. The clones were screened for recombinant protein expression by immunofluorescence staining and ELISA. The first method relied on the assumption that the higher the expression level, the more protein there is also accumulated on the intracellular secretory pathway. For ELISA, 96-well plates were coated with cell culture supernatants, and ELISA was performed with a monoclonal antibody against the SRCR domain of MARCO (for truncated MARCO-clones) or with polyclonal antibodies against the C-terminus of SCARA5. The highest expressors were expanded, and used for protein production. Zeocin selection was decreased to 250 μg/ml after the initial selection.

### Example 6

### Example 6. Production of Used HIS-Tagged cSRs in HEK-293 EBNA Cells

Selected cell lines excreting the His-tagged protein into cell culture media were grown as monolayers in DMEM with Glutamax-1 supplemented with 10% fetal calf serum (FCS), Penicillin-Streptomycin (PS) and sodium pyruvate (this is referred to herein as normal media). The cell culture media also included an appropriate antibiotic for selection (250 μg/ml G418 for maintenance of EBNA cells, 125 μg/ml zeosin, or 3 μg/ml puromycin) and daily addition of 100 μg/ml of ascorbic acid (only for proteins containing collagenous sequences). Upon confluency, the media was harvested and the cell debris was spun down (1000 rpm, 5 minutes). The cleared media was stored at +4° C. or −20° C. until purification. One of the cell plates was further split 1:4 to 1:6 and grown to confluency in DMEM.

In the other plates, the harvested media was replaced with serum-free DMEM/F-12 (1:1) supplemented with Glutamax-1, PS, sodium pyruvate, and ascorbic acid. This serum-free cell culture media was harvested after 3 days of culture. If possible, the cells were overlaid with fresh serum-free media for further protein production.

### Example 7

### Example 7. Purification of Used HIS-Tagged cSRs

The cell culture media was filtered and injected into a column packed with Talon-matrix equilibrated with 1× phosphate buffer (50 mM sodium phosphate, 150 mM NaCl, pH 7.0). After injection, the unbound proteins were washed with 2× phosphate buffer containing 10-15 mM imidazole. For elution of the His-tagged proteins, imidazole concentration was increased to 200 mM. After the elution, fractions containing the protein of interest were identified with SDS-PAGE, pooled and dialyzed against PBS to get rid of imidazole (3×2 hrs, 1000× volume of buffer). Separate purifications were made for samples with and without serum.

### Example 8

### Example 8. Generation of Affinity Media (API) Using the cSR from Example 1

In this set of experiments, Talon Superflow metal affinity resin (Clontech) was used as the solid phase to make an affinity medium. The capacity of the beads was between 5 to 20 mg protein/ml resin.

For the bacterial removal experiment, 1 ml of the resin was coupled with the chimeric protein of Example 1 purified from 2 liters of cell culture media (either from normal media (NM) or serum-free media (SF)). Two sets of beads were produced, one using the protein produced in NM, and the other using the protein produced in SF.

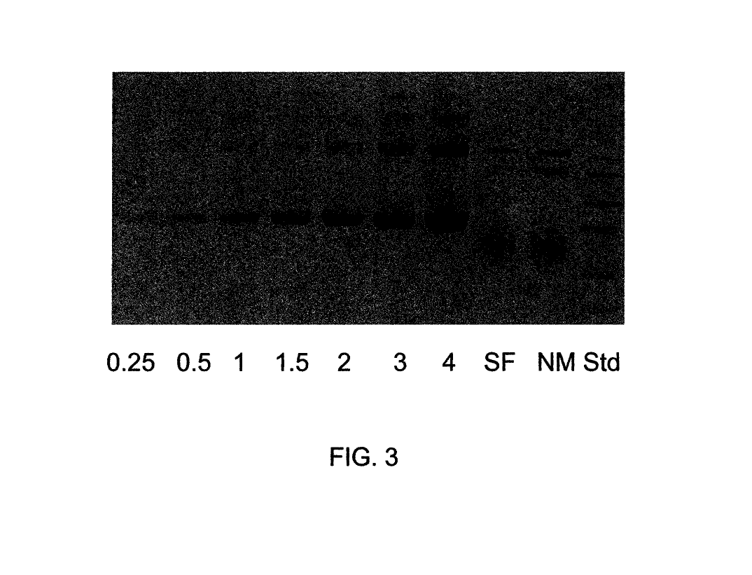

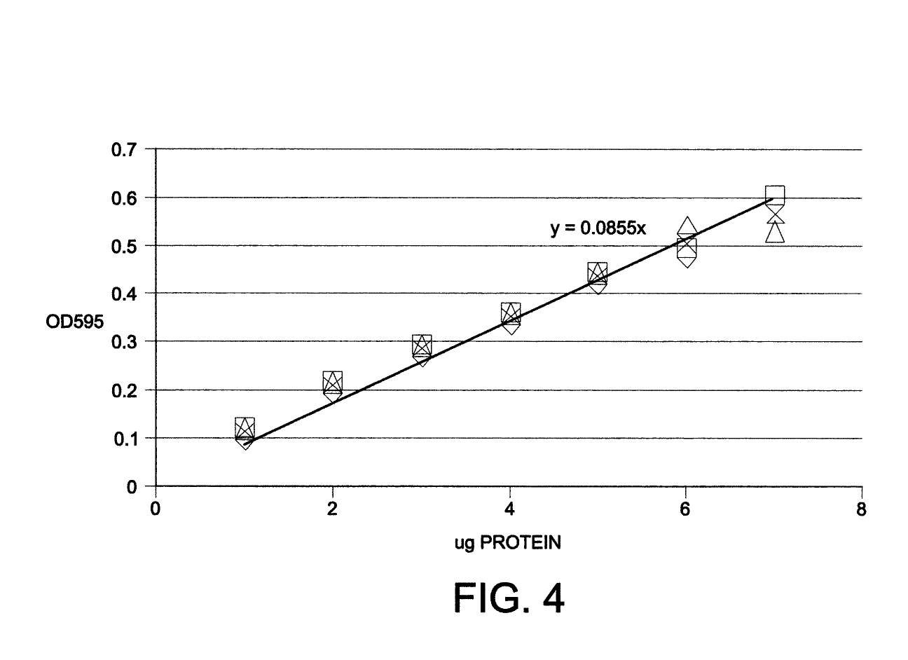

The purified chimeric protein dialyzed against PBS was mixed with the beads and rotated overnight at +4° C. After the binding, the beads were collected by running the suspension through a column by gravity. The beads were rinsed and suspended in 1:1 volume of PBS. A sample was taken from the suspension to analyze the amount of protein bound to the beads using two different methods: densitometry for stained protein bands on SDS-PAGE and Bradford assay for eluted proteins. 10 μl of beads was mixed with SDS loading buffer and a certain amount was loaded on a gel. In this example, 0.125 μl of beads were loaded on the gel. Based on the densitometry estimation (FIG. 3), the bound protein concentration was around 8 μg protein/μl beads. Based on the standard curve (FIG. 4) of a Bradford assay the concentrations of the eluted proteins were 2.1 (serum free) and 2.8 (normal media) μg/μl beads. OD595s for the eluted proteins were 0.178 and 0.239, respectively.

FIG. 3 shows the SDS-PAGE used for the densitometry estimation. Lanes 1-7 (from the left) included protein (BSA) at the marked concentration, in units of μg/lane. The lane marked SF used the chimeric protein from serum-free media. The lane marked NM used the chimeric protein from normal media. The lane marked Std indicates the molecular weight standards.

FIG. 4 shows the Bradford assay standard curve. The standard curve was made with triplicates of different BSA concentrations, and the equation was drawn based on the average. OD595 refers to the absorbance being measured at 595 nm.

### Example 9

### Example 9. Bacterial Removal Capacity of an Affinity Medium (API) Derived from Example 8

The coupled beads (NM and SF) and empty control beads were rinsed with PBS and incubated in human serum (which was not inactivated) to equilibrate the beads. The beads were rotated overnight at +4° C. The next day, four bacteria (ESBL E. coli, S. pneumoniae, multiple resistant S. aureus (MRSA), and N. meningitidis) were grown to OD=0.5 (≈3×10 exp 8 CFU/ml). The bacteria were diluted to ≈10 exp 3 CFU/ml in human serum and kept on ice. (Note: “10 exp n” is used herein to refer to 10 to the nth power.)

For each bacterial suspension, a control amount of 500 μl was incubated for 10 minutes on a shaker at +37° C. A viable count was performed on this control.

500 μl of the bacterial suspension was then added to 100 μl of coated beads in 5 ml columns and incubated for 10 minutes on a shaker at +37° C. The beads were spun down (1000 rpm, 1 min) and the bacterial suspension was pushed through the beads (Fraction 1). The beads were then washed 5× with 200 μl of PBS, 1×500 μl PBS, and 2×500 μl of 200 mM imidazole pH 8 in PBS (Fractions 2-9). Finally, the beads were resuspended in 500 μl of PBS (Fraction 10). Viable counts were performed for each fraction and plotted on a curve. This was performed for each of the three beads (NM, SF, and control).

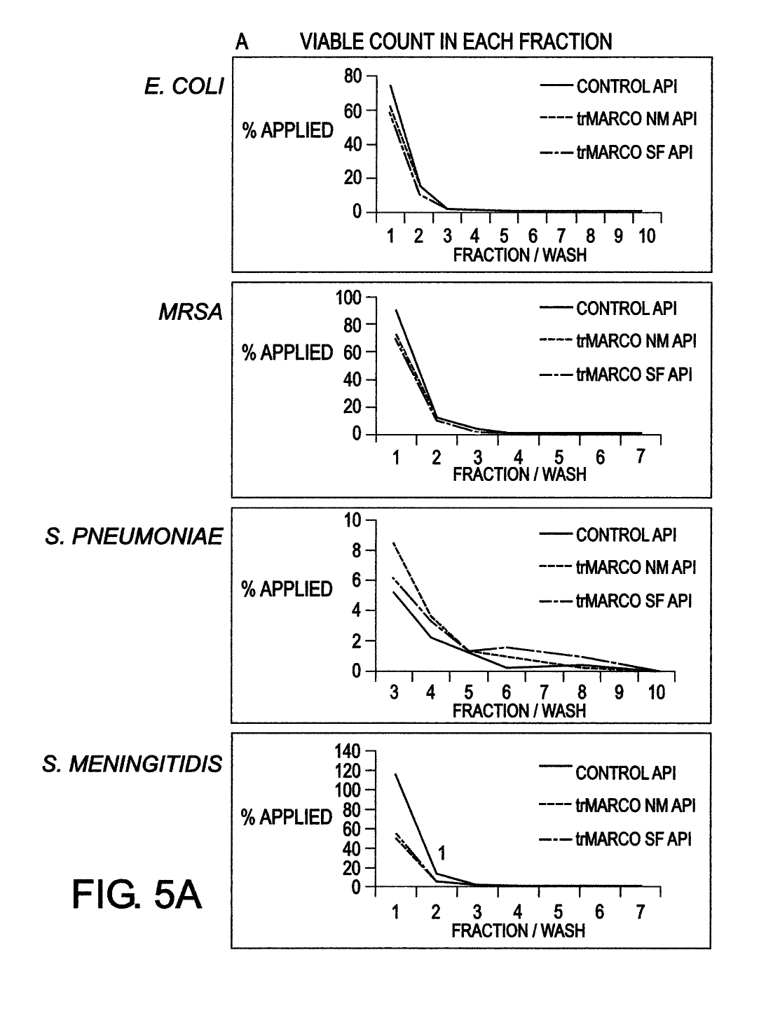

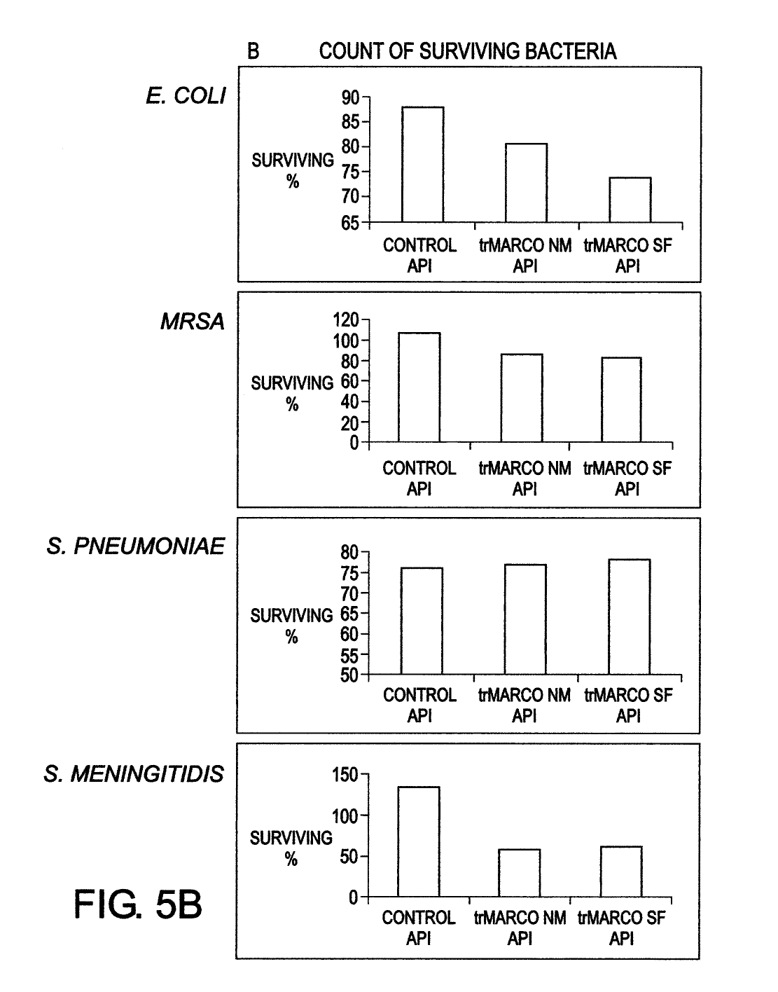

The results are shown in FIG. 5 separately for each bacteria and for each set of beads. There are four rows, corresponding to the bacteria. The left column provides the viable count in each fraction. The y-axis in these graphs is % applied, with 100% corresponding to the viable count in the control. The x-axis is the fraction. Note that the y-axis is different in each graph. The blue line is the control beads. The red line is the NM beads. The green line is the SF beads. These graphs in the left column essentially indicate how quickly the bacteria can be washed off the beads, which is a measure of how tightly the chimeric protein binds to the bacteria. As indicated by how the lines descend toward zero, the E. coli, MRSA, and N. meningitidis are washed in a similar rate from the beads, while the S. pneumoniae are removed slower from the SF or NM beads. This can be interpreted as the chimeric protein binding very strongly to S. pneumonia. As indicated by the y-axis, the % applied is higher for the control beads for E. coli, MRSA, and N. meningitides, indicating that fewer bacteria were killed by the control beads.

The right column provides the count of surviving bacteria in the 500 μl suspension. The y-axis in these graphs is % surviving, with 100% corresponding to all of the bacteria that were viable in the suspension prior to pushing the suspension through the beads. The bars represent the amount (%) of bacteria still present after the beads were removed (assuming the viable count for the control also applies to these suspensions). Note that the y-axis is different in each graph. For E. coli, the chimeric proteins removed a much higher percentage of the viable bacteria from the suspension than the control, with the SF cultured proteins removing almost 50% more than the NM cultured proteins. For MRSA, the chimeric proteins removed more of the bacteria than the control. For S. pneumoniae, the differences between the three beads were not that significant. For N. meningitidis, the chimeric proteins removed a large percentage of the bacteria compared to the control.

### Example 10

### Example 10. Bacterial Removal Capacity of an Affinity Medium (API) Using cSRs from Example 2

Chimeric protein was produced, purified and coupled to the beads as described in Examples 6-8. This chimeric protein contained a SR-A PRM with a SCARA5 anchor domain. The bound protein concentration was measured to be 4.2 μg/μl beads based on densitometry and 1.5 μg/μl beads based on Bradford analysis. A bacteria removal assay was performed as described in Example 9. However, in this experiment 200 μl of coated beads were used. Control beads were pre-incubated with human serum.

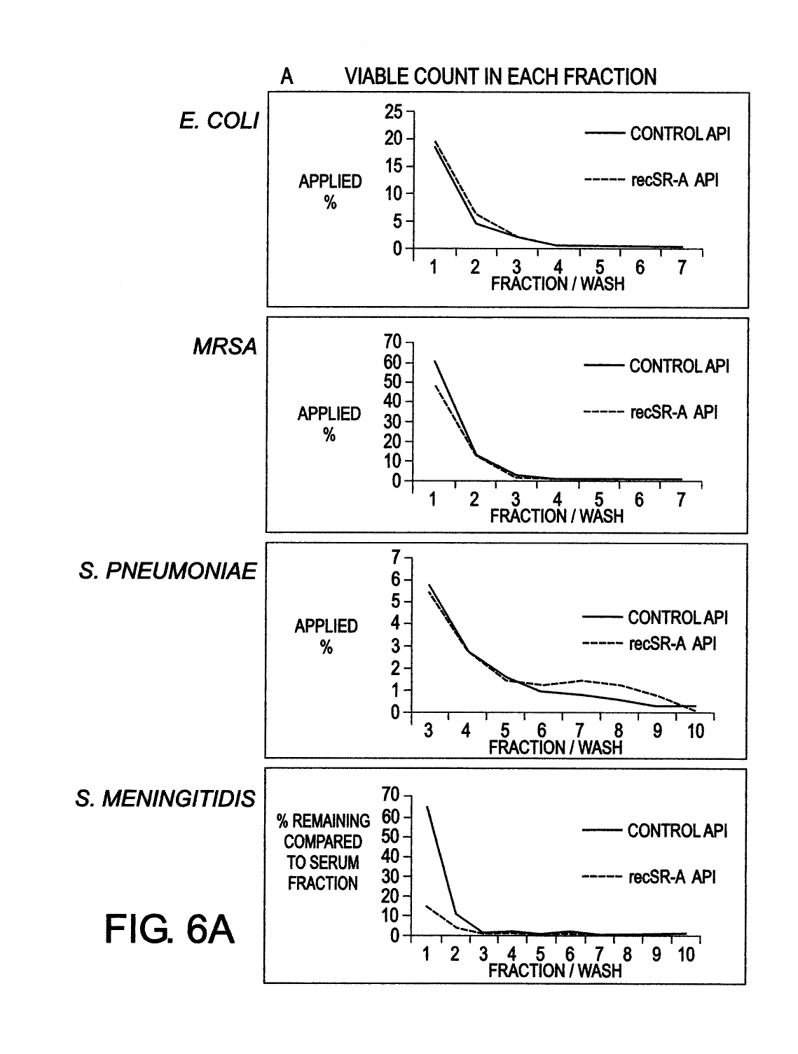

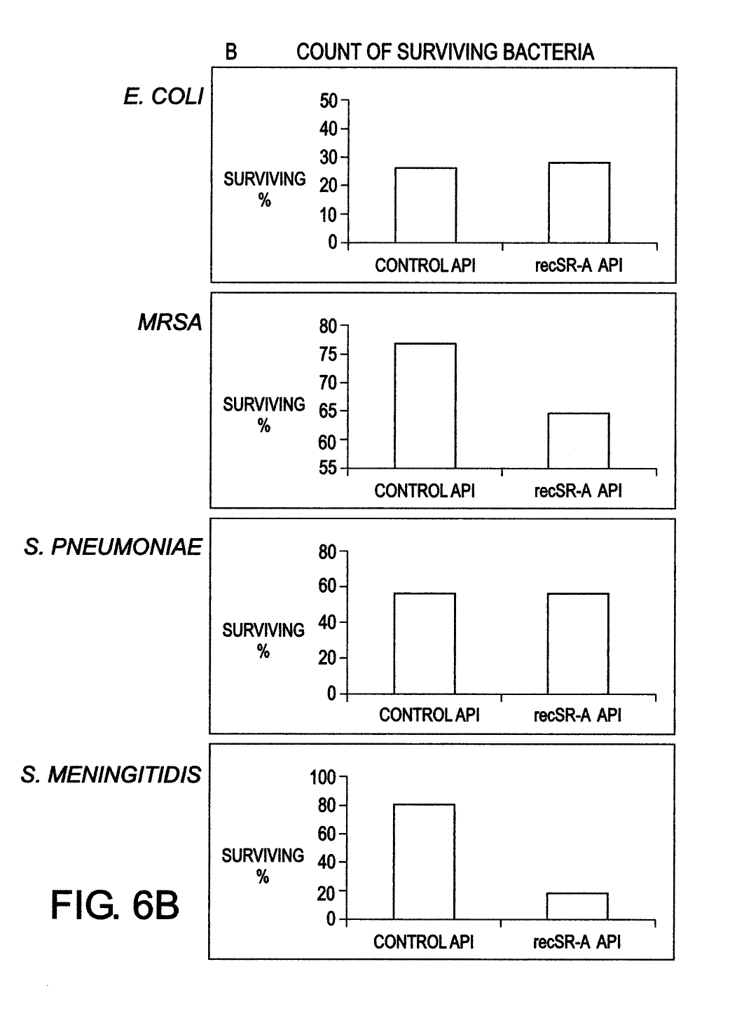

The results are shown in FIG. 6 separately for each bacteria and for each set of beads. Again, there are four rows, corresponding to the bacteria. The left column provides the viable count in each fraction. The y-axis in these graphs is % applied, with 100% corresponding to the viable count in the control. The x-axis is the fraction. Note that the y-axis is different in each graph. The blue line is the control beads. The red line is the beads with chimeric protein. Again, the S. pneumoniae were removed very slowly. This can be interpreted as the chimeric protein binding very strongly to S. pneumonia. As indicated by the y-axis, the % applied is higher for the control beads against MRSA and N. meningitides, indicating that of the bacteria removed, fewer of them were killed by the control beads.

The right column provides the count of surviving bacteria in the 500 μl suspension. The y-axis in these graphs is % surviving, with 100% corresponding to all of the bacteria that were viable in the suspension prior to pushing the suspension through the beads. The bars represent the amount (%) of bacteria still present after the beads were removed (assuming the viable count for the control also applies to these suspensions). Note that the y-axis is different in each graph. For MRSA and N. meningtidis, the chimeric proteins removed a much higher percentage of the bacteria than the control. The amount of removed bacteria was roughly the same for E. coli and S. pneumoniae.

### Example 11

### Example 11. Bacterial Removal Capacity of an Affinity Medium (API) Using cSRs from Example 3

Chimeric protein was produced, purified and coupled to the beads as described in Examples 6-8. This chimeric protein contained a SCARA5 anchor domain. The protein concentration on the beads was estimated to be 0.9 μg/μl beads.

The coupled beads and empty control beads were packed into a column with 2 ml bedvolume of matrix and rinsed with PBS. Next day, ESBL E. coli were grown to OD=0.5 (=3×10 exp 8 CFU/ml). The bacteria were diluted to 10 exp 4 CFU/ml in PBS and kept on ice.

A viable count was performed on this suspension and 1 ml was pushed into the column (Fraction 1). The elution was started with 2×1 ml of PBS (Fractions 2 and 3) to get rid of the void buffer volume in the column. The beads were then washed 3× with 300 μl of PBS (Fractions 4-6) containing most of the unbound bacteria. Thereafter the washes were continued 10× with 100 μl PBS (Fractions 7-16) showing the binding and/or killing of the bacteria in the cSR coated column. Finally the column was washed 20× with 300 μl of PBS (Fractions 17-36). Finally, the beads were resuspended in 1 ml of PBS to see the amount of viable bacteria left in the column (Fraction 37). Viable counts were performed for each fraction and plotted on a curve. This was performed for the control beads and cSR coated beads.

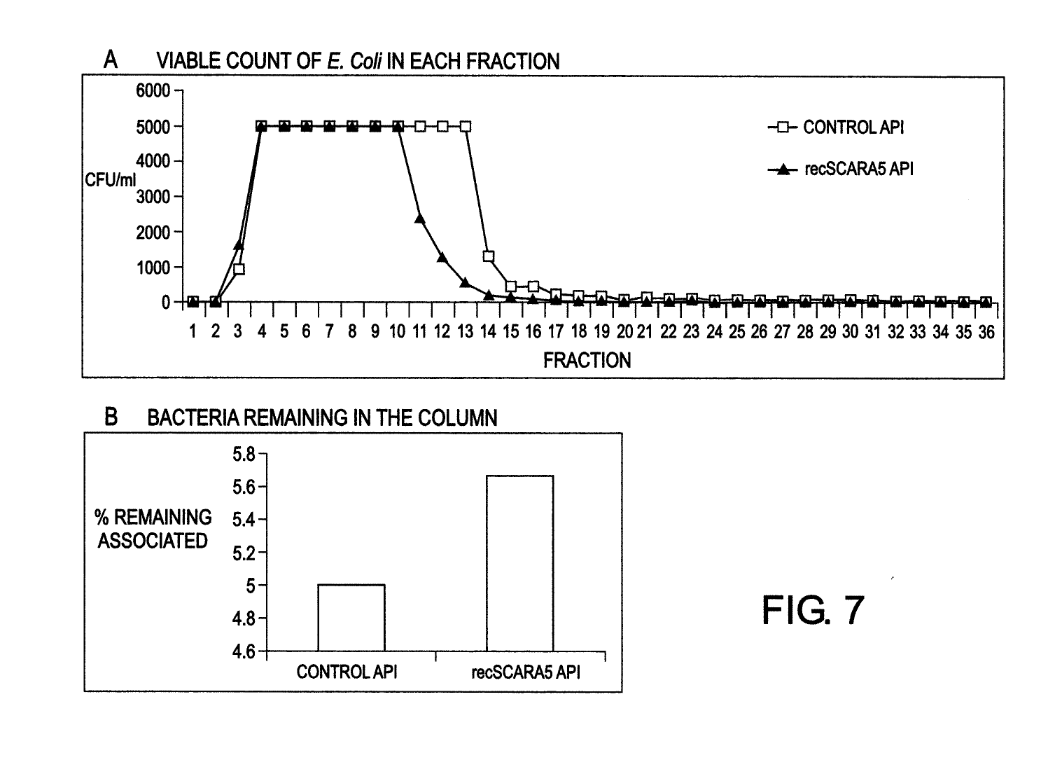

The results are shown in FIG. 7 for each set of beads. The left column provides the viable count in each fraction. The y-axis in these graphs is colony forming units (CFU)/ml. The x-axis is the fraction. The red line is the control beads. The green line is the beads with chimeric protein. The bacteria stop eluting earlier from the column with chimeric protein, indicating that more bacteria are bound or killed during the experiment.

The right column provides the count of surviving bacteria in the bead suspension. The y-axis in these graphs is % surviving, with 100% corresponding to all of the bacteria that were viable in the suspension prior to pushing the suspension through the beads. The amount of E. coli remaining in the column with chimeric protein is slightly higher.

### Example 12

### Example 12. Bacterial Removal Capacity of Affinity Medium (API) Using cSR from Example 4

Chimeric protein was produced, purified and coupled to the beads as described in Examples 6-8. This chimeric protein contained a truncated SCARA5 anchor domain. The protein concentration on the beads was estimated to be 1 μg/μl beads.

The coupled beads and empty control beads were packed into a column with 1 ml bedvolume of matrix and rinsed with PBS. Next day, ESBL E. coli were grown to OD=0.5 (=3×10 exp 8 CFU/ml). The bacteria were diluted to 10 exp 3 CFU/ml in non-inactivated human serum and kept on ice.

The bacterial suspension was incubated for 2 minutes at +37° C. A viable count was performed on this material prior to the next step.

Before applying the bacterial suspension to the columns, the columns were rinsed with human serum. Thereafter, 500 μl of the bacterial suspension was incubated for 2 min at +37° C. and pushed into the column. The elution was started with 2×300 μl of PBS (Fraction 1-2) to get rid of the void buffer volume in the column. The beads were then washed 12× with 100 μl of PBS (Fractions 3-14) showing the binding of the bacteria in the coated column. Finally, the beads were resuspended in 1 ml of PBS to see the amount of viable bacteria left in the column. Viable counts were performed for each fraction and plotted on a curve. This was performed for the control beads and chimeric protein coated beads.

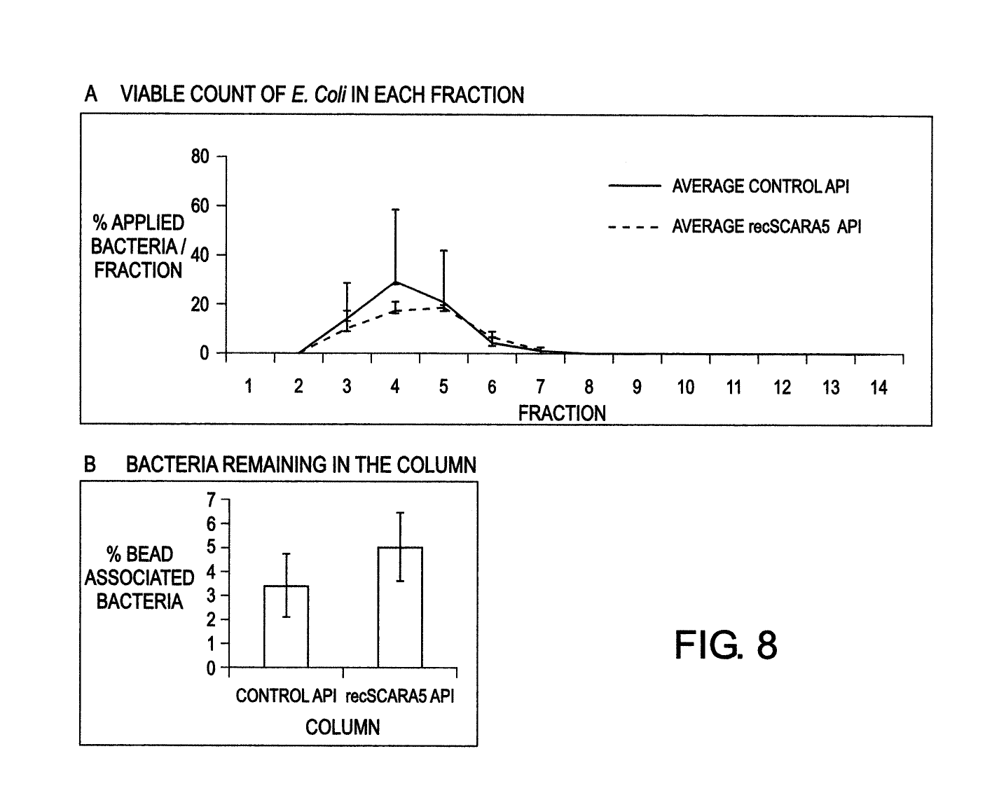

The results are shown in FIG. 8 for each set of beads. The left column provides the viable count in each fraction. The y-axis in these graphs is % of the concentration applied, with 100% corresponding to the viable count in the control. The x-axis is the fraction. The red line is the average of control beads. The purple line is the average of beads with chimeric protein. The number of bacteria eluting from the column with recombinant protein is slightly higher, indicating that less bacteria are bound or killed during the experiment in the test columns than the controls.

The right column provides the count of surviving bacteria in the bead suspension. The y-axis in these graphs is % surviving, with 100% corresponding to all of the bacteria that were viable in the suspension prior to adding them to the beads. In contrast to the data shown in the left column, the amount of E. coli remaining in the column with chimeric protein is slightly higher.

### Example 13

### Example 13. Immunocompetence in PBMCs Exposed to the Flow-Through Fractions from Examples 1 and 2

The fractions obtained in Examples 9 and 10 were used to treat peripheral blood mononuclear cells (PBMCs). The isolated PBMCs were plated (10 exp 5 cells/well) and treated next day for 4 hours with the eluted fractions and gentamycin to prevent bacterial growth. The cell culture media was collected and the amount of produced TNF-alpha was measured by ELISA using lipopolysaccharide (LPS) as a positive control. This experiment showed whether the fractions passing though the column resulted in immunocompetence of PBMCs

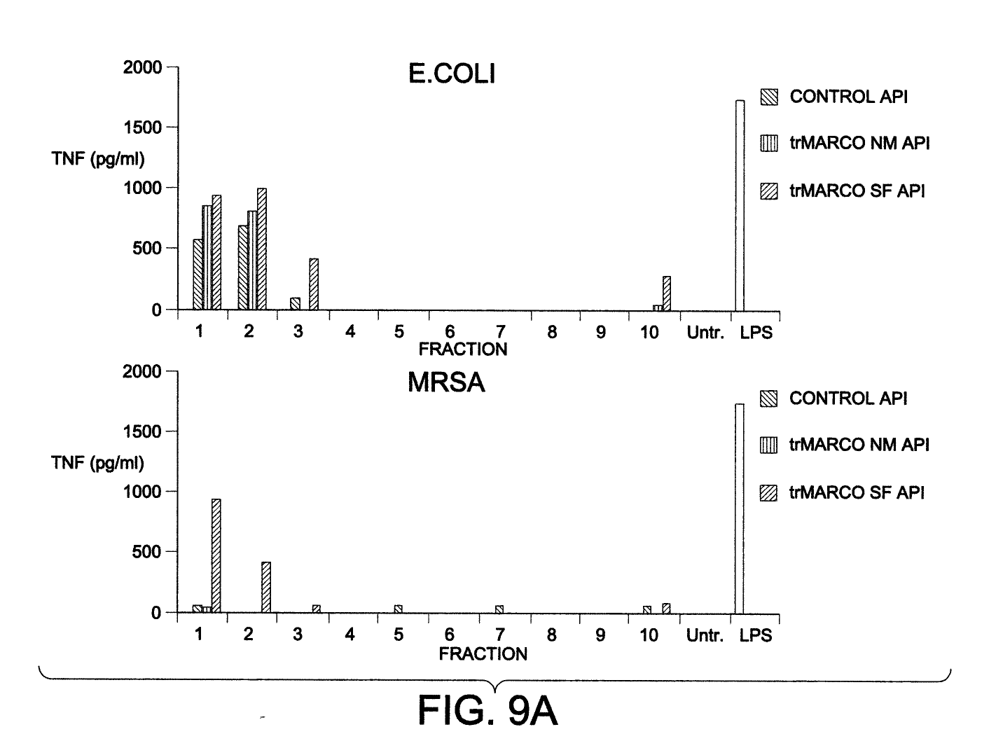

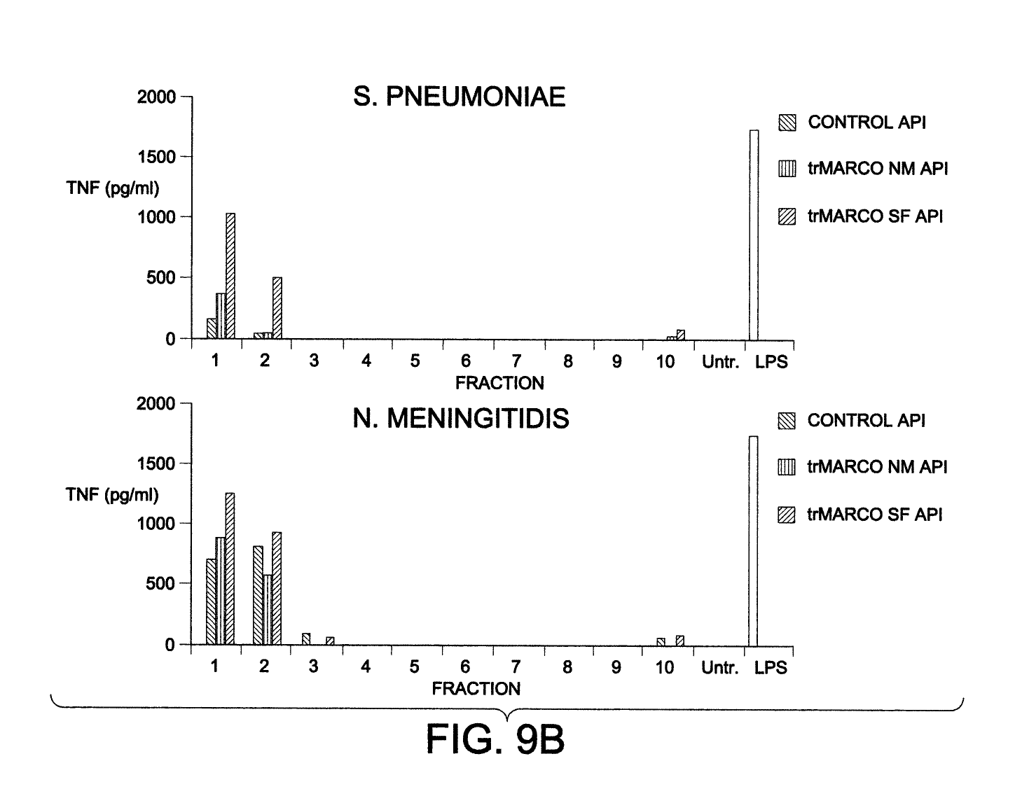

Results for the chimeric protein of Example 1 (PRM from MARCO plus MARCO anchor) are shown in FIG. 9. The y-axis is the amount of TNF-alpha, in pg/ml. The green line is the control. The purple line is the NM beads. The blue line is the SF beads. Fraction 10 here was the bacterial suspension remaining in the column after all the washes. As compared to the control beads, the beads with cSRs showed higher activation for all of the bacteria in Fraction 10 (i.e. binding to the beads), as well as in the first fractions (containing free bacteria). The preserved immunoresponse suggests that a higher amount of immune activating molecules were found in fractions from cSR columns, and this together with the lower live bacteria counts (FIG. 5 and FIG. 6), suggests that more bacteria were destroyed in these.

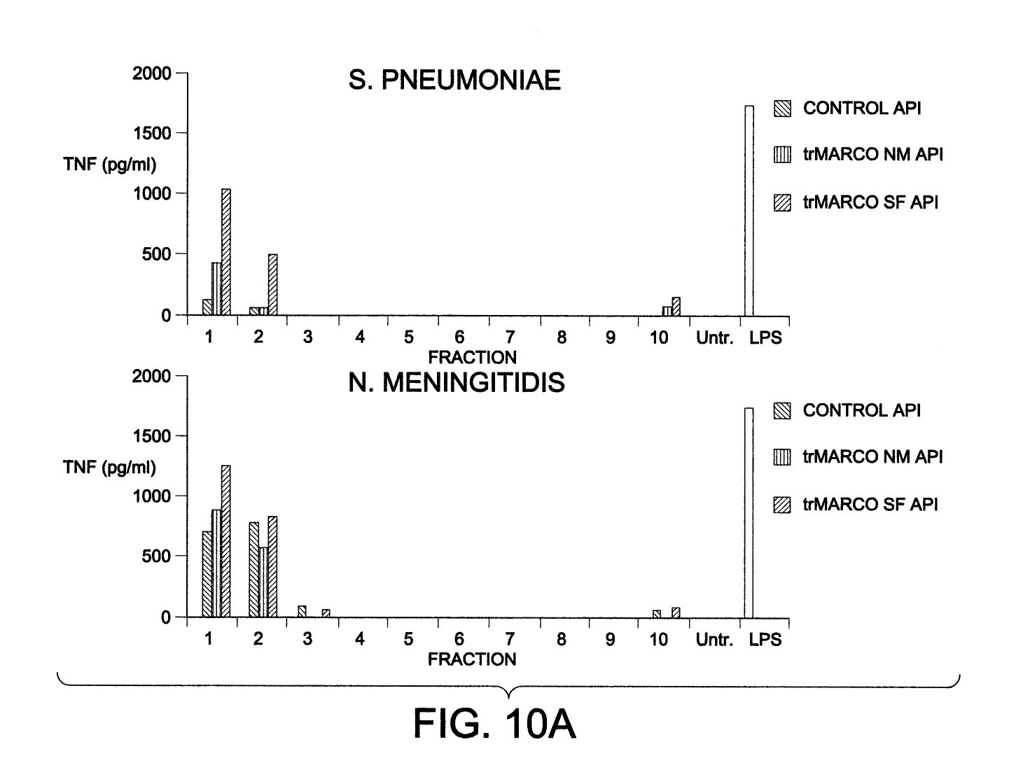

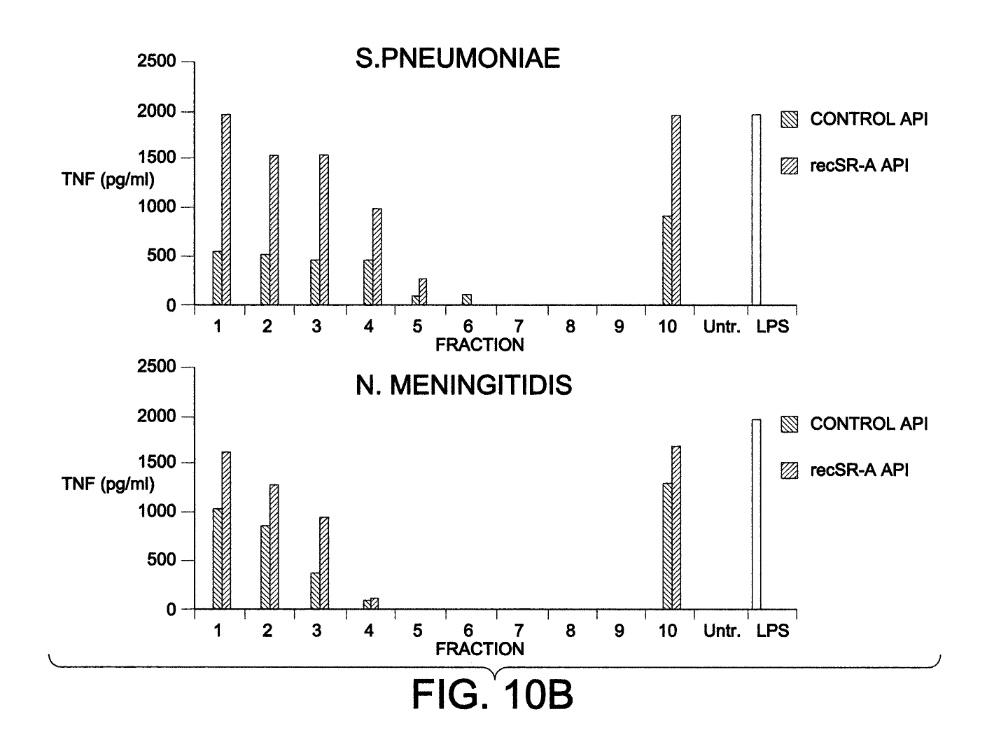

Results for the cSR of Example 2 (PRM from SR-A plus SCARA5 anchor) are shown in FIG. 10. The green line is the control. The purple line is the beads with chimeric protein. Again, Fraction 10 here was the bacterial suspension remaining in the column after all the washes. As compared to the control beads, the beads with chimeric protein showed higher activation for all of the bacteria in Fraction 10 (i.e. binding to the beads) and all but E. coli in the first fractions (containing free bacteria).

### Example 14

### Example 14: Binding of E. coli and acLDL by Transmembrane Forms of SCARA5, the cSR of Example 2 (SCARA5 Anchor with a SCARA1 SRCR Domain as the PRM), and a cSR Made by Combining the SCARA5 Anchor with a SCARA2 SRCR Domain as the PRM

All three proteins were transfected into CHO cells and expressed. For comparison, control cells had no protein expressed. Each cell type was then exposed to heat-killed E. coli and acetylated LDL to determine binding. An anti-SCARA5 antibody was used to demonstrate the presence of expressed proteins.

The results are shown in FIG. 11. The control cells bound weakly to all three materials. Cells with intact SCARA5 bound the E. coli and weakly bound AcLDL. The SCARA5/SCARA2 cSR strongly bound both E. coli and AcLDL. The SCARA5/SCARA1 cSR also strongly bound E. coli and AcLDL. Comparing the receptors, the two cSRs exhibited higher binding of the ligands than did intact SCARA5, demonstrating how SR binding affinity can be improved by the present disclosure.

### Example 15

### Example 15: Internalization of Bacterial LPS by SCARA5

Mouse SCARA5 transfected CHO cells were examined for internalization of LPS. Crumbs 2 (Crb2) expressing CHO cells were used as a negative control. Actin staining (red) was used to indicate the presence of cells. The results are shown in FIG. 12. The row marked “Ab staining” indicates successful transfection and expression (green staining obtained with antibodies recognizing SCARA5 and Crb2). The row marked “LPS internalization” indicates intracellular LPS (green) after endocytosis. These results demonstrate for the first time that SCARA5 is able to bind to and internalize LPS.

### Example 16

### Example 16: Differences in Affinity for Bacteria Between Chimeric Receptors

The chimeric receptor proteins (APIs) of SEQ ID NO: 41, SEQ ID NO: 43, and SEQ ID NO: 35 were expressed and purified, and then coupled to beads (Talon®, Clontech, U.S.A.) as described in Example 8. As a control, empty beads with no protein were used.

Both the beads coupled with chimeric receptors and the empty control beads were rinsed with PBS and then incubated in human serum (not inactivated) overnight at +4° C. The next day, four bacteria (ESBL E. coli, S. pneumoniae, multiple resistant S. aureus (MRSA), and biotinylated/heat-killed MRSA) were grown to OD=0.5 (≈3×10 exp 8 CFU/ml). The bacteria were diluted to ≈10 exp 3 CFU/ml in human serum and kept on ice.

For the control, 100 μl of empty beads was incubated with 500 μl of human serum for 10 minutes on a shaker at +37° C. A viable count was performed on this control.

500 μl of the bacterial suspension was then added to 100 μl of coated beads in 5 ml columns and incubated for 10 minutes on a shaker at +37° C. The beads were spun down (1000 rpm, 1 min) and the bacterial suspension was pushed through (Fraction 1). The beads were then washed 5× with 200 μl of PBS, then 1×500 μl PBS (Fractions 2-7). Finally, the beads were resuspended in 500 μl of PBS (Fraction 8).

The chimeric protein of SEQ ID NO: 35 was also biotinylated and bound to a Streptavidin-coated bead matrix (Pierce Chemical Corp., U.S.A.). This biotinylated protein was tested only against MRSA.

Viable counts were performed for each fraction and plotted on a curve, and samples of each fraction were also used in a PBMC stimulation assay (described above). All the preceding steps were performed separately for each of the tested APIs.

Table 1 shows the relative drop in viability for selected pathogens following passage through a column containing beads coupled to the different chimeric receptor proteins as indicated. The colony counts obtained from the column flow-through is expressed as a percentage of the colony count obtained from the same stock prior to passage through the column. Biotinylated, dead multi-resistant S. aureus (MRSA) served as a negative control throughout.

TABLE 1

| | Relative count of surviving bacteria (%) | | | | |
|---|---|---|---|---|---|
| | | | | | Biotinylated |
| | | SEQ ID | SEQ ID | SEQ ID | SEQ ID |
| Bacteria | Control | NO: 35 | NO: 43 | NO: 41 | NO: 35 |
| E. coli | 100 | 76 | 95 | 90 | — |
| S. pneumoniae | 100 | 86 | 90 | 77 | — |
| MRSA | 100 | 76 | 95 | 90 | 86 |
| Biotinylated | 0 | 0 | 0 | 0 | — |
| MRSA | | | | | |

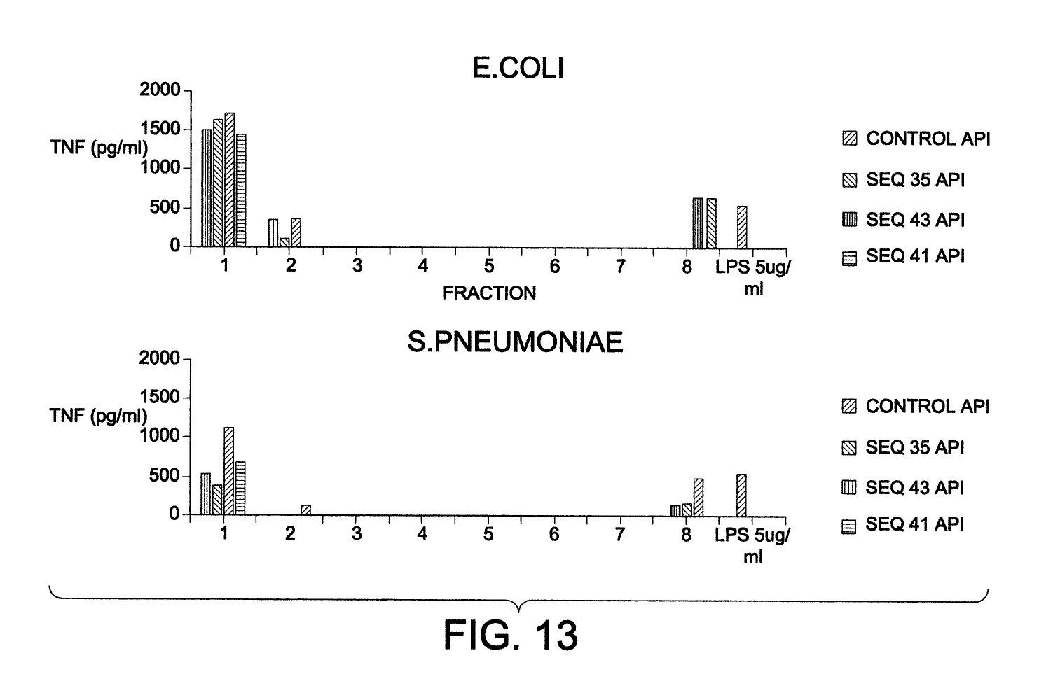

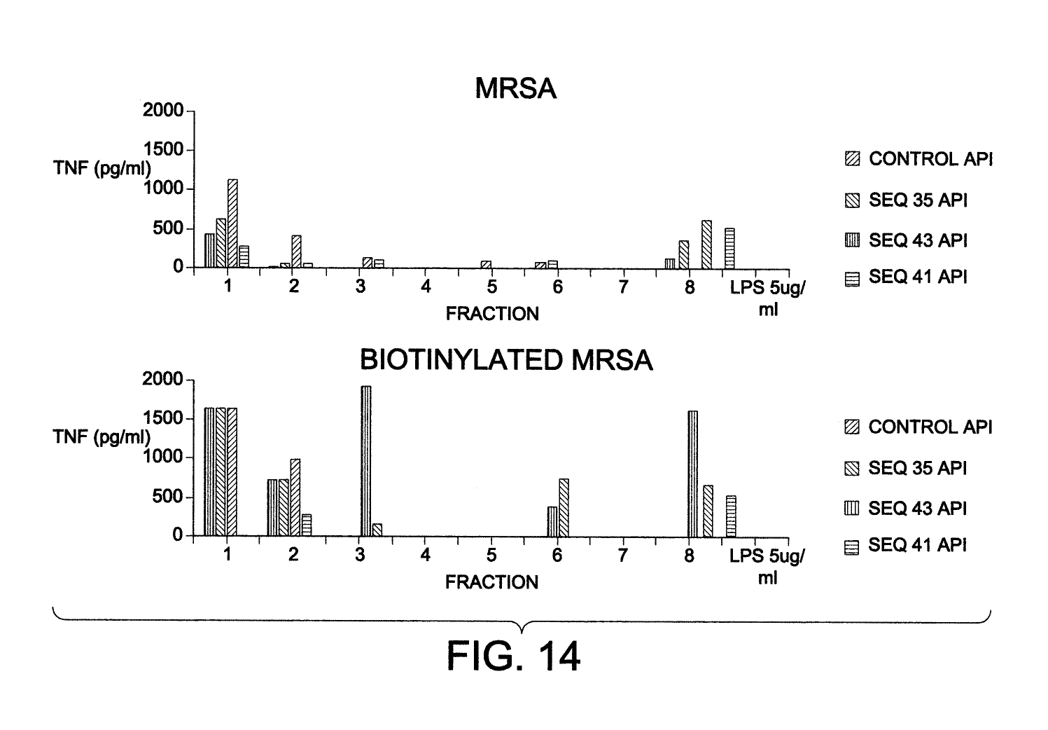

FIG. 13 and FIG. 14 are graphs showing the results of the PBMC assay, expressed as units of TNF and compared to 5 μg/mL LPS as well as the control. Each graph essentially indicates how quickly the bacteria can be washed off the beads, which is a measure of how tightly the given chimeric protein binds to the bacteria. As indicated by how the PBMCs respond with increased TNF production in the last fraction (8), significant numbers of E. coli, MRSA (living and biotinylated/heat-killed), and S. pneumoniae remain on the beads containing chimeric proteins also after washing, suggesting a relatively strong binding.

This can be interpreted as the chimeric protein binding very strongly to S. pneumoniae. Furthermore, for S. pneumonia, a small non-specific binding to the control matrix is also observed after washing.

As indicated by the pattern of TNF responses for different APIs, the binding affinities are different for each bacterial species and each API.

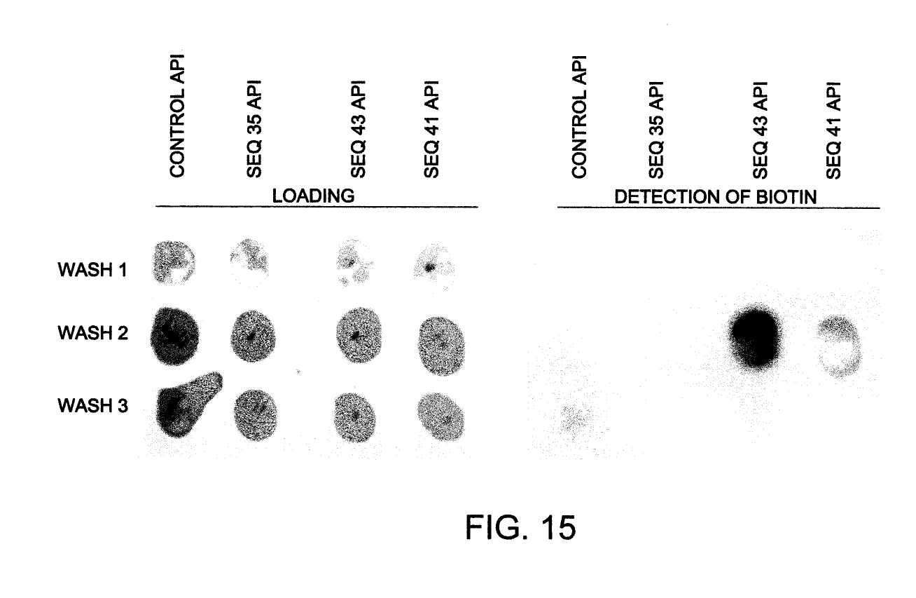

FIG. 15 shows the results of dot blots performed using an anti-biotin antibody on fluids after a column containing 100 microliters (μl) of Streptavidin-coated beads coupled with the biotinylated chimeric proteins of SEQ ID NO: 41, SEQ ID NO: 43, or SEQ ID NO: 35, or empty control beads was washed with different solutions. In each case, the beads were rinsed with PBS and then incubated in human serum (not inactivated) overnight at +4° C. Multi-resistant S. aureus (MRSA) were grown to OD=0.5 (≈3×10 exp 8 CFU/ml) and diluted in human serum to ≈10 exp 3 CFU/ml and kept on ice. The bacteria were biotinylated following the manufacturer's protocol. After the biotinylation, bacteria were killed by boiling and the experiment performed using the protocol described for live bacteria in Example 13. Next, and in addition to the washes with PBS, three different wash solutions were tested. The beads were washed three times, first with 1×500 μl of 500 mM NaCl (Wash 1), next with 1× 500 μl of pH 5 MES, 500 mM NaCl (Wash 2), and finally with 1×500 μl of pH 3 glycine (Wash 3). For each column and wash, 4 μl of eluate was pipetted onto a blotting membrane and incubated with the primary antibody (HRP-conjugated strepavidin). The left panel shows the total loaded onto each column, while the right pane shows if and when bound ligands could be eluted.

### Example 17

### Example 17. Cell Transfection Results

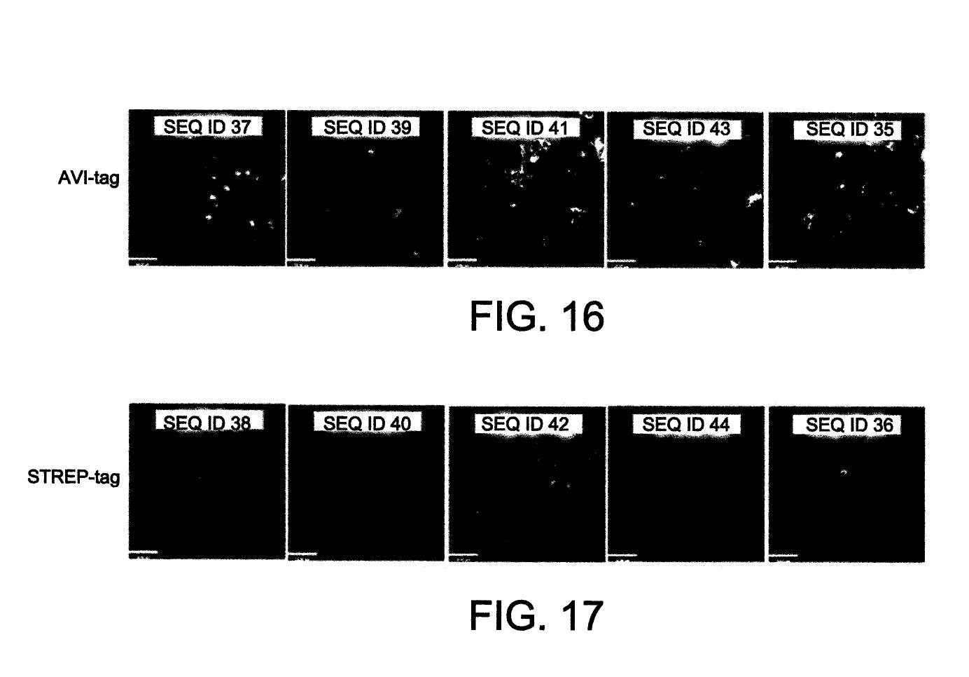

HEK-EBNA cultures were transfected as previously described. The cultures were transfected with five different chimeric scavenger receptor proteins. For each chimeric proteins, two different sequences were prepared that used different affinity tags, either the AviTag™ (SEQ ID NO: 45) or STREP (SEQ ID NO: 31) in addition to penta-His tag (SEQ ID NO: 28). After 48 hours, the cultures were stained using penta-His antibody (Qiagen) staining and immunohistochemistry pictures were taken. Those pictures are shown in FIG. 16 and FIG. 17, which are arranged in a row and column format.

FIG. 16 shows the pictures for the sequences using the AviTag™. From left to right, the cultures were transfected with SEQ ID NO: 37, SEQ ID NO: 39, SEQ ID NO: 41, SEQ ID NO: 43, and SEQ ID NO: 35. FIG. 17 shows the pictures for the sequences using the STREP tag. From left to right, the cultures were transfected with SEQ ID NO: 38, SEQ ID NO: 40, SEQ ID NO: 42, SEQ ID NO: 44, and SEQ ID NO: 36. The results should be compared by column between the two figures, i.e. compare the result of SEQ ID NO: 37 with SEQ ID NO: 38, compare SEQ ID NO: 39 with SEQ ID NO: 40, etc.

Differences in staining indicate variations in protein production levels. In general, cells expressing sequences incorporating the AviTag™ (in FIG. 16) attained higher production than cells expressing sequences containing STREP (in FIG. 17). The choice of the linker, affinity tag, and/or anchor domain is seen to affect the ability to successfully express the recombinant chimeric protein in sufficient quantities.

### Example 18

### Example 18: Ligand Binding does not Affect Immobilization on Solid Phase

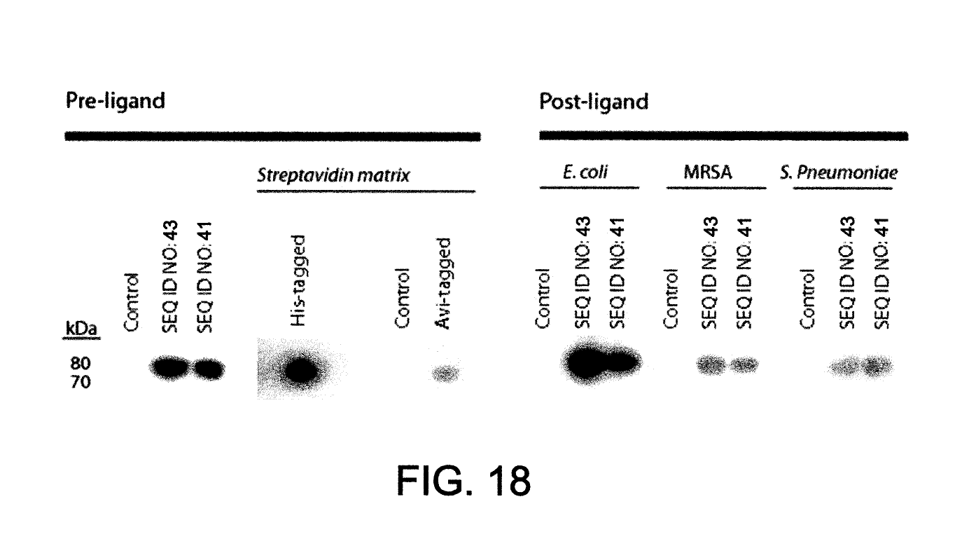

Beads containing directionally bound proteins expressed using SEQ ID NO: 41 or SEQ ID NO: 43, along with the AviTag™ (SEQ ID NO: 45) were prepared as described in Example 8 above, along with naked Talon beads or Streptavidin-coated agarose beads. In FIG. 18, the section labeled “Pre-ligand” shows how aliquots of both cSRs were either directly applied to gels for Western blotting using an anti-biotin antibody, or allowed to incubate with one of two matrixes (Talon matrix for His-tagged proteins or Strepavidin-coated beads for Avi-tagged, biotinylated proteins), drained and the flow-through then applied to the same gel. The control lane consisted of empty beads (no proteins).

Also in FIG. 18, the section labeled “Post-treatment” shows the same batch of beads and protein undergoing a similar procedure as above, with the addition of a pre-coupling step before coupling to beads and entailing mixing free cSRs with, respectively, E. coli, MRSA, and S. pneumoniae prepared as described in Example 16. The same control was used.

These data demonstrate that binding to both ligand and solid phase are specific and may occur independently of one another.

The present disclosure has been described with reference to exemplary embodiments. Obviously, modifications and alterations will occur to others upon reading and understanding the preceding detailed description. It is intended that the present disclosure be construed as including all such modifications and alterations insofar as they come within the scope of the appended claims or the equivalents thereof.

1. A recombinant chimeric protein comprising:
a pathogen recognition module from a mammalian scavenger receptor; and
an anchor domain.

2. The protein of claim 1, further comprising a linker between the pathogen recognition module and the anchor domain.

3. The protein of claim 2, wherein the linker is a sequence of about 2 to about 15 amino acids.

4. The protein of claim 2, wherein the linker includes at least two different amino acids.

5. The protein of claim 4, wherein the at least two different amino acids are selected from the group consisting of alanine, histidine, and glycine.

6. The protein of claim 1, further comprising an affinity tag.

7. The protein of claim 6, wherein the affinity tag is selected from the group consisting of hemagglutinin, AviTag, V5, Myc, T7, FLAG, HSV, VSV-G, 6-His, biotin, and STREP.

8. The protein of claim 1, wherein the pathogen recognition module is C-terminal and the anchor domain is from a mammalian scavenger receptor.

9. The protein of claim 8, wherein the pathogen recognition module is from a mammalian class A scavenger receptor.

10. The protein of claim 1, wherein the anchor domain is an Fc region of an immunoglobulin.

11. The protein of claim 1, wherein the anchor domain is from a mammalian scavenger receptor different than the pathogen recognition module.

12. The protein of claim 1, wherein the anchor domain is an alpha-helical coiled coil domain from a mammalian class A scavenger receptor.

13. The protein of claim 8, wherein the pathogen recognition module is a SRCR domain, a collagenous domain E, a cysteine-rich domain, a C-type lectin-like domain (CTLD), a lysosomal-associated membrane protein (LAMP)-like domain, a mucin-like domain, or an EGF-like domain.

14. The protein of claim 1, further comprising a signal peptide.

15. A nucleic acid sequence encoding the protein of claim 1.

16. A vector comprising a nucleic acid sequence encoding the protein of claim 1.

17. A host cell including a vector comprising a nucleic acid sequence encoding the protein of claim 1.

18. (canceled)

19. (canceled)

20. A method for treating a disease associated with a pathogen or a pathogen-derived molecule, comprising:
receiving an affinity medium comprising a solid phase and a chimeric protein immobilized upon the solid phase, the chimeric protein comprising (i) a pathogen recognition module from a scavenger receptor and (ii) an anchor domain;
contacting a biological sample with the affinity medium to bind the pathogen or pathogen-derived molecule to the affinity medium.

21. (canceled)

22. (canceled)

23. (canceled)

24. A recombinant chimeric protein, consisting of:
a signal peptide;
at least one affinity tag;
a linker;
a pathogen recognition module from a mammalian scavenger receptor; and
an anchor domain.

25. The recombinant chimeric protein of claim 24, wherein the pathogen recognition module and the anchor domain from different mammalian scavenger receptors.

26. (canceled)

27. (canceled)
